(19) United States
(12) Patent Application Publication
(10) Pub. No.: US 2025/0312441 A1
(43) Pub. Date: Oct. 9, 2025

(54) IMMUNOGENIC COMPOSITIONS FOR HERPES SIMPLEX VIRUS PROTEINS

(71) Applicant: MERCK SHARP & DOHME LLC, Rahway, NJ (US)

(72) Inventors: Constantine James KONSTANTOULAS, Vienna (AT); Sabrina SCHRAUF, Mörbisch am See (AT); Dai WANG, Blue Bell, PA (US)

(73) Assignee: MERCK SHARP & DOHME LLC, Rahway, NJ (US)

(21) Appl. No.: 18/874,243

(22) PCT Filed: Jun. 22, 2023
(86) PCT No.: PCT/US2023/025920

§ 371 Date: Dec. 12, 2024

Related U.S. Application Data
(60) Provisional application No. 63/354,852, filed on Jun. 23, 2022.

(51) Int. Cl. A61K 39/245; A61P 37/04; C07K 14/005; C12N 15/86

(52) U.S. Cl. CPC A61K 39/245; A61P 37/04; C07K 14/005; C12N 15/86; C12N 2710/16622; C12N 2710/16634; C12N 2760/18443

(57) ABSTRACT

The disclosure provides live attenuated measles vectors encoding in their genome one or more heterologous genes for herpes simplex virus (HSV) proteins, e.g., glycoprotein B, glycoprotein C, glycoprotein D, and variants thereof, as well as nucleic acid constructs encoding such measles vectors. The disclosure also relates to immunogenic compositions comprising live attenuated measles vectors encoding HSV proteins, immunogenic compositions comprising such measles vectors, and use of such measles vectors and immunogenic compositions to induce an immune response to HSV in subjects.

### FIELD

This disclosure relates generally to viral vector-based immunogenic compositions against Herpes Simplex Virus.

### BACKGROUND OF THE INVENTION

Herpes simplex viruses (HSV) are double-stranded linear DNA viruses in the Herpesviridae family. Two members of the herpes simplex virus family infect humans, known as HSV-1 and HSV-2. Symptoms of HSV infection include the formation of blisters in the skin or mucous membranes of the mouth, lips, and/or genitals. HSV is a neuro-invasive virus that can cause sporadic recurring episodes of viral reactivation in infected individuals. HSV is transmitted by contact with an infected area of the skin during a period of viral activation. Despite a primed immune system, reactivation of the virus is frequent, often leading to lesions at the original site of infection.

The World Health Organization estimated that in 2022, 491 million people worldwide were infected with HSV-2, and 3.7 billion people under the age of 50 worldwide were infected with HSV-1. HSV-2 infection results in an approximately 3-fold increase in the risk of acquiring HIV. However, no HSV vaccine currently exists. Accordingly, there is a need for a vaccine that can reduce the prevalence of HSV infection.

### SUMMARY OF THE INVENTION

In a first aspect, the disclosure provides an isolated nucleic acid molecule comprising: a) a cDNA encoding a full length, antigenomic (+) RNA strand of an attenuated strain of measles virus (MV-cDNA); b) one or more cDNAs encoding an herpes simplex virus (HSV) protein (HSV cDNA) selected from the group consisting of: gC, gD, gB, UL19, and variants thereof; c) an upstream additional transcriptional unit (ATU) cDNA operably linked to the HSV cDNA that is 5′ of the HSV cDNA (upstream ATU cDNA); and d) a downstream ATU cDNA operably linked to the HSV cDNA that is 3′ of the HSV cDNA (downstream ATU cDNA); wherein the upstream ATU cDNA, the HSV cDNA, and the downstream ATU cDNA are between P and M genes of the MV-cDNA at ATU2 or between H and L genes of the MV-cDNA at ATU3.

In some embodiments of the first aspect, each of the one or more HSV cDNAs encodes an HSV protein sequence independently selected from the group consisting of: SEQ ID NO: 54 (HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO: 58.1 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19). In some embodiments of the first aspect, the upstream ATU cDNA, the HSV cDNA, and the downstream ATU cDNA is at ATU2 in the MV-cDNA. In some embodiments of the first aspect, the upstream ATU cDNA, the HSV cDNA, and the downstream ATU cDNA is at ATU3 in the MV-cDNA. In some embodiments of the first aspect, the upstream ATU cDNA sequence is set forth in SEQ ID NO: 87. In some embodiments of the first aspect, the downstream ATU cDNA sequence is set forth in SEQ ID NO: 90. In some embodiments of the first aspect, the isolated nucleic acid molecule comprises a sequence selected from the group consisting of SEQ ID NOs: 59-63 and 96-97. In some embodiments of the first aspect, the isolated nucleic acid molecule comprises a sequence set forth in SEQ ID NO: 59. In some embodiments of the first aspect, the isolated nucleic acid molecule consists of a sequence set forth in SEQ ID NO: 59. In some embodiments of the first aspect, the isolated nucleic acid molecule comprises a sequence set forth in SEQ ID NO: 60. In some embodiments of the first aspect, the isolated nucleic acid molecule consists of a sequence set forth in SEQ ID NO: 60. In some embodiments of the first aspect, the isolated nucleic acid molecule comprises a sequence set forth in SEQ ID NO: 61. In some embodiments of the first aspect, the isolated nucleic acid molecule consists of a sequence set forth in SEQ ID NO: 61. In some embodiments of the first aspect, the isolated nucleic acid molecule comprises a sequence set forth in SEQ ID NO: 62. In some embodiments of the first aspect, the isolated nucleic acid molecule consists of a sequence set forth in SEQ ID NO: 62. In some embodiments of the first aspect, the isolated nucleic acid molecule comprises a sequence set forth in SEQ ID NO: 63. In some embodiments of the first aspect, the isolated nucleic acid molecule consists of a sequence set forth in SEQ ID NO: 63.

In a second aspect, the disclosure provides an isolated nucleic acid molecule comprising: a) a cDNA encoding a full length, antigenomic (+) RNA strand of an attenuated strain of measles virus (MV-cDNA); b) a first cDNA encoding an herpes simplex virus (HSV) protein selected from the group consisting of: gC, gD, gB, UL19, and variants thereof (first HSV cDNA); c) a second cDNA encoding an HSV protein selected from the group consisting of: gC, gD, gB, UL19, and variants thereof (second HSV cDNA), wherein the first and second HSV cDNAs do not have the same sequence; d) an upstream additional transcriptional unit (ATU) cDNA that is 5′ of the first HSV cDNA (upstream ATU cDNA); e) a downstream ATU cDNA that is 3′ of the second HSV cDNA (downstream ATU cDNA); and f) an interstitial ATU cDNA between the first and second HSV cDNAs (interstitial ATU cDNA); wherein the upstream ATU cDNA, the first and second HSV cDNAs, the interstitial ATU cDNA and the downstream ATU cDNA are operably linked; and wherein the upstream ATU cDNA, the first and second HSV CDNAs, the interstitial ATU cDNA, and the downstream ATU cDNA are between P and M genes of the MV-cDNA at ATU2 or between H and L genes of the MV-cDNA at ATU3.

In some embodiments of the second aspect, the first and second HSV cDNAs each encode an HSV protein sequence independently selected from the group consisting of: SEQ ID NO: 54 (HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO: 100 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19). In some embodiments of the second aspect, the upstream ATU cDNA, the first and second HSV cDNAs, and the downstream ATU cDNA are at ATU2 in the MV-cDNA. In some embodiments, the upstream ATU cDNA, the first and second HSV cDNAs, and the downstream ATU cDNA are at ATU3 in the MV-cDNA. In some embodiments of the second aspect, the upstream ATU cDNA sequence is set forth in SEQ ID NO: 87. In some embodiments of the second aspect, the downstream ATU cDNA sequence is set forth in SEQ ID NO: 90. In some embodiments of the second aspect, the interstitial ATU cDNA sequence is selected from the group consisting of SEQ ID NOs: 83, 87, 90, and 92. In some embodiments of the second aspect, the isolated nucleic acid molecule comprises a sequence selected from the group consisting of SEQ ID NOs: 66, 68, 69, and 70. In some embodiments of the second aspect, the isolated nucleic acid molecule comprises a sequence set forth in SEQ ID NO: 66. In some embodiments of the second aspect, the isolated nucleic acid molecule consists of a sequence set forth in SEQ ID NO: 66. In some embodiments of the second aspect, the isolated nucleic acid molecule comprises a sequence set forth in SEQ ID NO: 68. In some embodiments of the second aspect, the isolated nucleic acid molecule consists of a sequence set forth in SEQ ID NO: 68. In some embodiments of the second aspect, the isolated nucleic acid molecule comprises a sequence set forth in SEQ ID NO: 69. In some embodiments of the second aspect, the isolated nucleic acid molecule consists of a sequence set forth in SEQ ID NO: 69. In some embodiments of the second aspect, the isolated nucleic acid molecule comprises a sequence set forth in SEQ ID NO: 70. In some embodiments of the second aspect, the isolated nucleic acid molecule consists of a sequence set forth in SEQ ID NO: 70.

In a third aspect, the disclosure provides an isolated nucleic acid molecule comprising: a) a cDNA encoding a full length, antigenomic (+) RNA strand of an attenuated strain of measles virus (MV-cDNA); b) a first cDNA encoding an herpes simplex virus (HSV) protein (HSV cDNA) selected from the group consisting of: gC, gD, gB, UL19, and variants thereof (first HSV cDNA); c) a second HSV cDNA encoding an HSV protein selected from the group consisting of: gC, gD, gB, UL19, and variants thereof (second HSV cDNA), wherein the first and second HSV cDNAs do not have the same sequence; d) an upstream additional transcriptional unit (ATU) cDNA that is 5′ of the first HSV cDNA (upstream ATU cDNA); e) a downstream ATU cDNA that is 3′ of the second HSV cDNA (downstream ATU cDNA); and f) a furin cleavage site cDNA and 2A peptide cDNA between the first and second HSV cDNAs (Fur-2A cDNA); wherein the upstream ATU cDNA, the first and second HSV CDNAs, the Fur-2A cDNA, and the downstream ATU cDNA are operably linked; and wherein the upstream ATU cDNA, the first and second HSV cDNAs, the Fur-2A cDNA, and the downstream ATU cDNA are between P and M genes of the MV-cDNA at ATU2 or between H and L genes of the MV-cDNA at ATU3.

In some embodiments of the third aspect, the first and second HSV cDNAs each encode an HSV protein sequence independently selected from the group consisting of: SEQ ID NO: 54(HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO: 100 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19). In some embodiments of the third aspect, the upstream ATU cDNA, the first and second HSV cDNAs, the Fur-2A cDNA, and the downstream ATU cDNA are at ATU2 in the MV-cDNA. In some embodiments of the third aspect, the upstream ATU cDNA, the first and second HSV cDNAs, the Fur-2A cDNA, and the downstream ATU cDNA are at ATU3 in the MV-cDNA. In some embodiments of the third aspect, the upstream ATU cDNA sequence is set forth in SEQ ID NO: 87. In some embodiments of the third aspect, the downstream ATU cDNA sequence is set forth in SEQ ID NO: 90. In some embodiments of the third aspect, the furin cDNA of the Fur-2A cDNA encodes a protein sequence selected from the group consisting of SEQ ID NOs: 14-53, and wherein the 2A peptide cDNA of the Fur-2A cDNA encodes a protein sequence selected from the group consisting of SEQ ID NOs: 4-11.

In a fourth aspect, the disclosure provides an isolated nucleic acid molecule comprising: a) a cDNA encoding a full length, antigenomic (+) RNA strand of an attenuated strain of measles virus (MV-cDNA); b) a first cDNA encoding an herpes simplex virus (HSV) protein selected from the group consisting of: gC, gD, gB, UL19, and variants thereof (first HSV cDNA); c) a second HSV cDNA encoding an HSV protein selected from the group consisting of: gC, gD, gB, UL19, and variants thereof (second HSV cDNA); d) a third HSV cDNA encoding an HSV protein selected from the group consisting of: gC, gD, gB, UL19, and variants thereof (third HSV cDNA), wherein the first, second, and third HSV cDNAs do not have the same sequence; e) an upstream additional transcriptional unit (ATU) cDNA that is 5′ of the first HSV cDNA (upstream ATU cDNA); f) a downstream ATU cDNA that is 3′ of the third HSV cDNA (downstream ATU cDNA); g) a first interstitial ATU cDNA between the first and second HSV protein cDNAs (first interstitial ATU cDNA); h) a second interstitial ATU cDNA between the second and third HSV CDNAs (second interstitial ATU cDNA); wherein the upstream ATU cDNA, the first, second, and third HSV cDNAs, the first and second interstitial ATU cDNAs, and the downstream ATU cDNA are operably linked; and wherein the upstream ATU cDNA, the first, second, and third HSV cDNAs, the first and second interstitial ATU cDNAs, and the downstream ATU cDNA are between P and M genes of the MV-cDNA at ATU2 or between H and L genes of the MV-cDNA at ATU3.

In some embodiments of the fourth aspect, the first, second, and third HSV CDNAs each encode an HSV protein sequence independently selected from the group consisting of: SEQ ID NO: 54 (HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO: 100 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19). In some embodiments of the fourth aspect, the upstream ATU cDNA, the first, second, and third HSV cDNAs, and the downstream ATU cDNA are at ATU2 in the MV-cDNA. In some embodiments of the fourth aspect, the upstream ATU cDNA, the first, second, and third HSV cDNAs, and the downstream ATU cDNA are at ATU3 in the MV-cDNA. In some embodiments of the fourth aspect, the upstream ATU cDNA sequence is set forth in SEQ ID NO: 87. In some embodiments of the fourth aspect, the downstream ATU cDNA sequence is set forth in SEQ ID NO: 90. In some embodiments of the fourth aspect, the first and second interstitial ATU cDNA sequences are independently selected from the group consisting of SEQ ID NOs: 83, 87, 90, and 92. In some embodiments of the fourth aspect, the isolated nucleic acid molecule comprises a sequence selected from the group consisting of SEQ ID NOs: 71, 75, 76, 79, and 80. In some embodiments of the fourth aspect, the isolated nucleic acid molecule comprises a sequence set forth in SEQ ID NO: 71. In some embodiments of the fourth aspect, the isolated nucleic acid molecule consists of a sequence set forth in SEQ ID NO: 71. In some embodiments of the fourth aspect, the isolated nucleic acid molecule comprises a sequence set forth in SEQ ID NO: 75. In some embodiments of the fourth aspect, the isolated nucleic acid molecule consists of a sequence set forth in SEQ ID NO: 75. In some embodiments of the fourth aspect, the isolated nucleic acid molecule comprises a sequence set forth in SEQ ID NO: 76. In some embodiments of the fourth aspect, the isolated nucleic acid molecule consists of a sequence set forth in SEQ ID NO: 76. In some embodiments of the fourth aspect, the isolated nucleic acid molecule comprises a sequence set forth in SEQ ID NO: 79. In some embodiments of the fourth aspect, the isolated nucleic acid molecule consists of a sequence set forth in SEQ ID NO: 79. In some embodiments of the fourth aspect, the isolated nucleic acid molecule comprises a sequence set forth in SEQ ID NO: 80. In some embodiments of the fourth aspect, the isolated nucleic acid molecule consists of a sequence set forth in SEQ ID NO: 80.

In a fifth aspect, the disclosure provides an isolated nucleic acid molecule comprising: a) a cDNA encoding a full length, antigenomic (+) RNA strand of an attenuated strain of measles virus (MV-cDNA); b) a first cDNA encoding an herpes simplex virus (HSV) protein selected from the group consisting of: gC, gD, gB, UL19, and variants thereof (first HSV cDNA); c) a second HSV cDNA encoding an HSV protein selected from the group consisting of: gC, gD, gB, UL19, and variants thereof (second HSV cDNA); d) a third HSV cDNA encoding an HSV protein selected from the group consisting of: gC, gD, gB, UL19, and variants thereof (third HSV cDNA), wherein the first, second, and third HSV cDNAs do not have the same sequence; e) an upstream additional transcriptional unit (ATU) cDNA that is 5′ of the first HSV cDNA (upstream ATU cDNA); f) a downstream ATU cDNA that is 3′ of the third HSV cDNA (downstream ATU cDNA); g) a first furin cleavage site cDNA and 2A peptide cDNA between the first and second HSV cDNAs (first Fur-2A cDNA); and h) a second furin cleavage site cDNA and 2A peptide cDNA (second Fur-2A cDNA) between the second and third HSV cDNAs (second Fur-2A cDNA); wherein the upstream ATU cDNA, the first, second, and third HSV cDNAs, the first and second Fur-2A cDNAs, and the downstream ATU cDNA are operably linked; and wherein the upstream ATU cDNA, the first, second, and third HSV cDNAs, the first and second Fur-2A cDNAs, and the downstream ATU cDNA are between P and M genes of the MV-cDNA at ATU2 or between H and L genes of the MV-cDNA at ATU3.

In some embodiments of the fifth aspect, the first and second HSV cDNAs each encode an HSV protein sequence independently selected from the group consisting of: SEQ ID NO: 54 (HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO: 100 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19). In some embodiments of the fifth aspect, the upstream ATU cDNA, the first, second, and third HSV cDNAs, the first and second Fur-2A cDNAs, and the downstream ATU cDNA are at ATU2 in the MV-cDNA. In some embodiments of the fifth aspect, the upstream ATU cDNA, the first, second, and third HSV cDNAs, the first and second Fur-2A cDNAs, and the downstream ATU cDNA are at ATU3 in the MV-cDNA. In some embodiments of the fifth aspect, the upstream ATU cDNA sequence is set forth in SEQ ID NO: 87. In some embodiments of the fifth aspect, the downstream ATU cDNA sequence is set forth in SEQ ID NO: 90. In some embodiments of the fifth aspect, the furin cDNA of the first and second Fur-2A cDNAs encodes a protein sequence independently selected from the group consisting of SEQ ID NOs: 14-53, and wherein the 2A peptide cDNA of the first and second Fur-2A cDNAs is independently selected from the group consisting of SEQ ID NOs: 4-11. In some embodiments of the fifth aspect, the isolated nucleic acid molecule comprises the sequence set forth in SEQ ID NO: 82. In some embodiments of the fifth aspect, the isolated nucleic acid molecule consists of the sequence set forth in SEQ ID NO: 82.

In a sixth aspect, the disclosure provides an isolated nucleic acid molecule comprising: a) a cDNA encoding a full length, antigenomic (+) RNA strand of an attenuated strain of measles virus (MV-cDNA); b) a first cDNA encoding an herpes simplex virus (HSV) protein selected from the group consisting of: gC, gD, gB, UL19, and variants thereof (first HSV cDNA); c) a second HSV cDNA encoding an HSV protein selected from the group consisting of: gC, gD, gB, UL19, and variants thereof (second HSV cDNA); d) a third HSV cDNA encoding an HSV protein selected from the group consisting of: gC, gD, gB, UL19, and variants thereof (third HSV cDNA), wherein the first, second, and third HSV cDNAs do not have the same sequence; e) an upstream additional transcriptional unit (ATU) cDNA that is 5′ of the first HSV cDNA (upstream ATU cDNA); f) a downstream ATU cDNA that is 3′ of the third HSV cDNA (downstream ATU cDNA); g) a furin cleavage site cDNA and 2A peptide cDNA (Fur-2A cDNA); and h) an interstitial ATU cDNA; wherein the upstream ATU cDNA, the first, second, and third HSV CDNAs, the Fur-2A cDNA, the interstitial ATU cDNA, and the downstream ATU cDNA are operably linked; wherein i) the Fur-2A cDNA is between the first and second HSV cDNAs and the interstitial ATU cDNA is between the second and third HSV cDNAs, or ii) the interstitial ATU cDNA is between the first and second HSV cDNAs and the Fur-2A cDNA is between the second and third HSV cDNAs; and wherein the upstream ATU cDNA, the first, second, and third HSV cDNAs, the Fur-2A cDNA, the interstitial ATU cDNA, and the downstream ATU cDNA are between P and M genes of the MV-cDNA at ATU2 or between H and L genes of the MV-cDNA at ATU3.

In some embodiments of the sixth aspect, the first and second HSV cDNAs each encode an HSV protein sequence independently selected from the group consisting of: SEQ ID NO: 54(HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO: 100 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19). In some embodiments of the sixth aspect, the upstream ATU cDNA, the first, second, and third HSV cDNAs, the Fur-2A cDNA, the interstitial ATU cDNA, and the downstream ATU cDNA are at ATU2 in the MV-cDNA. In some embodiments of the sixth aspect, the upstream ATU cDNA, the first, second, and third HSV cDNAs, the Fur-2A cDNA, the interstitial ATU cDNA, and the downstream ATU cDNA are at ATU3 in the MV-cDNA. In some embodiments of the sixth aspect, the upstream ATU cDNA sequence is set forth in SEQ ID NO: 87. In some embodiments of the sixth aspect, the downstream ATU cDNA sequence is set forth in SEQ ID NO: 90. In some embodiments of the sixth aspect, the furin cDNA of the Fur-2A cDNA encodes a protein sequence selected from the group consisting of SEQ ID NOs: 14-53, and wherein the 2A peptide cDNA of the Fur-2A cDNA encodes a protein sequence selected from the group consisting of SEQ ID NOs: 4-11. In some embodiments of the sixth aspect, the interstitial ATU cDNA sequence is selected from the group consisting of SEQ ID NOs: 83, 87, 90, and 92. In some embodiments of the sixth aspect, the Fur-2A cDNA is between the first and second HSV cDNAs and the interstitial ATU cDNA is between the second and third HSV cDNAs. In some embodiments of the sixth aspect, the interstitial ATU cDNA is between the first and second HSV cDNAs and the Fur-2A cDNA is between the second and third HSV cDNAs. In some embodiments of the sixth aspect, the isolated nucleic acid molecule comprises a sequence selected from the group consisting of SEQ ID NOs: 73-74. In some embodiments of the sixth aspect, the isolated nucleic acid molecule comprises a sequence set forth in SEQ ID NO: 73. In some embodiments of the sixth aspect, the isolated nucleic acid molecule consists of a sequence set forth in SEQ ID NO: 73. In some embodiments of the sixth aspect, the isolated nucleic acid molecule comprises a sequence set forth in SEQ ID NO: 74. In some embodiments of the sixth aspect, the isolated nucleic acid molecule consists of a sequence set forth in SEQ ID NO: 74.

In a seventh aspect, the disclosure provides a vector for the rescue of a recombinant measles virus comprising the isolated nucleic acid molecule of any one of the above aspects and embodiments. In some embodiments of the seventh aspect, the vector comprises a CMV promoter. In some embodiments of the seventh aspect, the vector comprises the sequence set forth in SEQ ID NO: 81. In some embodiments of the seventh aspect, the vector consists of the sequence set forth in SEQ ID NO: 81. In some embodiments of the seventh aspect, the vector comprises a T7 promoter. In some embodiments of the seventh aspect, the vector comprises the sequence set forth in SEQ ID NO: 3.

In an eighth aspect, the disclosure provides a recombinant measles virus comprising in its genome a cDNA sequence comprising the nucleic acid molecule of any one of the above aspects and embodiments.

In a ninth aspect, the disclosure provides an immunogenic composition comprising (i) an effective amount of the recombinant measles virus of the eighth aspect, and (ii) a pharmaceutically acceptable carrier.

In a tenth aspect, the disclosure provides methods for treating or preventing a herpes simplex virus (HSV) infection in a subject in need thereof, comprising administering an effective amount of the immunogenic composition according to the ninth aspect to the subject.

In an eleventh aspect, the disclosure provides methods for inducing a protective immune response against herpes simplex virus (HSV) in a subject in need thereof, comprising administering an effective amount of the immunogenic composition of the ninth aspect to the subject.

In some embodiments of the tenth and eleventh aspects, the methods comprise a first administration of the immunogenic composition and a second administration of the immunogenic composition. In some embodiments, the protective immune response is a humoral immune response and/or a cellular immune response. In some embodiments, the second administration is performed from one month to two months after the first administration. In some embodiments, the subject is a human. In some embodiments of the tenth and eleventh aspects, the protective immune response against HSV prevents or reduces the severity of primary genital disease associated with HSV infection.

The disclosure provides uses of the recombinant measles virus of the eighth aspect or the immunogenic composition of the ninth aspect for preventing or treating an HSV infection in a subject in need thereof. The disclosure also provides for the use of the recombinant measles virus of the eighth aspect or the immunogenic composition of the ninth aspect for the manufacture of a medicament for the prevention or treatment of an HSV infection.

The disclosure also provides the recombinant measles virus of the eighth aspect or the immunogenic composition the ninth aspect, for use in preventing or treating an HSV infection in a subject in need thereof.

The disclosure also provides in vitro use of the recombinant measles virus of the eighth aspect or the immunogenic composition of the ninth aspect for expressing an HSV protein in eukaryotic cells.

In a twelfth aspect, the disclosure provides for an isolated peptide comprising the sequence set forth in SEQ ID NO: 58 (HSV-2 gBmut), SEQ ID NO: 100 (HSV-2 gBmutdel25), or SEQ ID NO: 64 (HSV-2 gBwtdel25), wherein SEQ ID NOs: 95 and 64 do not include residues 877-901 of SEQ ID NO: 55 (HSV-2 gB wild-type), or variants thereof, and wherein SEQ ID NOs: 58 and 95 comprise an alanine at position 665, an alanine at position 675, and an alanine at position 677.

In some embodiments of the twelfth aspect, the variant of SEQ ID NO: 58 has 95%, 96%, 97%, 98%, or 99% homology to the amino acid of SEQ ID NO: 58, the variant of SEQ ID NO: 100 has 95%, 96%, 97%, 98%, or 99% homology to the amino acid of SEQ ID NO: 100, or the variant of SEQ ID NO: 64 has 95%, 96%, 97%, 98%, or 99% homology to the amino acid of SEQ ID NO: 64. In some embodiments, the isolated polypeptide consists of SEQ ID NO: 58 (HSV-2 gBmut), SEQ ID NO: 100 (HSV-2 gBmutdel25), or SEQ ID NO: 64 (HSV-2gBwtdel25), wherein SEQ ID NOs: 95 and 64 do not include residues 877-901 of SEQ ID NO: 55 (HSV-2 gB wild-type).

In a thirteenth aspect, the disclosure provides an isolated nucleic acid molecule encoding the isolated peptide of any one of the isolated peptides of the twelfth aspect.

In a fourteenth aspect, the disclosure provides an immunogenic composition comprising (i) an effective amount of the isolated peptide of any one of the isolated peptide embodiments of the twelfth aspect, and (ii) a pharmaceutically acceptable carrier.

In a fifteenth aspect, the disclosure provides methods for treating or preventing a herpes simplex virus (HSV) infection in a subject in need thereof, comprising administering an effective amount of the immunogenic composition of the twelfth aspect.

In a sixteenth aspect, The disclosure provides methods for inducing a protective immune response against herpes simplex virus (HSV) in a subject in need thereof, comprising administering an effective amount of the immunogenic composition of the twelfth aspect to the subject.

In some embodiments of the fifteenth and sixteenth aspects, the methods comprise a first administration of the immunogenic composition and a second administration of the immunogenic composition. In some embodiments, the protective immune response is a humoral immune response and/or a cellular immune response. In some embodiments, the second administration is performed from one month to two months after the first administration. In some embodiments, the subject is a human. In some embodiments of the tenth and eleventh aspects, the protective immune response against HSV prevents or reduces the severity of primary genital disease associated with HSV infection.

The disclosure provides uses of the isolated peptides of any one of the isolated peptides of the twelfth aspect or the immunogenic composition of the fourteenth aspect for preventing or treating an HSV infection in a subject in need thereof.

The disclosure also provides the isolated peptides of any one of the embodiments of the twelfth aspect or the immunogenic composition of the fourteenth aspect, for use in preventing or treating an HSV infection in a subject in need thereof.

The disclosure also provides in vitro use of the isolated peptides of any one of the embodiments of the twelfth aspect or the immunogenic composition of the fourteenth aspect for expressing an HSV protein in eukaryotic cells.

The summary of the technology described above is non-limiting and other features and advantages of the technology will be apparent from the following detailed description, and from the claims.

### BRIEF DESCRIPTION OF THE DRAWINGS

FIG. 1 shows a schematic diagram of a recombinant measles vector genome. Additional transcriptional units (ATUs) are marked. ATU1 is positioned before the measles N gene; ATU2 is positioned between the measles P and M genes, and ATU3 is positioned between the measles H and L genes.

FIG. 2 shows line graphs comparing growth of MV expressing monovalent HSV-2 antigens (wild-type gB (gBwt), mutated gB (gBmut), wild-type gD (gD), or gC (F327A; gC) from the ATU2 or the ATU3 positions in MV. Virus release from cells and cell-associated virus were both measured (TCID50/mL).

FIG. 3A shows photographs of antigen expression from Vero cells infected by bivalent MV constructs B-08, B-09, and B-10. FIG. 3B shows photographs of antigen expression from Vero cells infected by trivalent MV constructs T-11 and T-15. FIG. 3C shows photographs of antigen expression from Vero cells infected by trivalent MV constructs T-12, T-13, T-14, and T-16.

FIG. 4A shows a graph of viral titer released from cells (TCID50/ml) across days post-infection for bivalent MV-HSV constructs as described in Example 4. FIG. 4B shows a graph of viral titer released from cells (TCID50/ml) across days post-infection for trivalent MV-HSV constructs as described in Example 4.

FIGS. 5A-5B show graphs of viral titer released from cells (FIG. 5A) and cell-associated virus (FIG. 5B) across days post-infection for trivalent MV-HSV constructs T-15 and T-18 in Example 5, specifically at late time points of infection. FIGS. 5C-5D show graphs of viral titer released from cells (FIG. 5C) and cell-associated virus (FIG. 5D) across days post-infection for trivalent MV-HSV constructs T-15 and T-18 in Example 5, specifically at early time points of infection.

FIG. 6 shows agarose gels of PCR-amplified HSV inserts from MV-HSV construct T-15 (gD_ATUa_gBwt_ATUb_gC) at passages 3, 4, 5, 6, and 7.

FIG. 7A shows gB antibody responses in cotton rats to various MV-HSV constructs and MV-Schwarz control virus at day 28, day 42, and day 49 after receiving two doses of a given construct. FIG. 7B shows gC antibody responses in cotton rats to various MV-HSV constructs and MV-Schwarz control virus at day 28, day 42, and day 49 after receiving two doses of a given construct. FIG. 7C shows gD antibody responses in cotton rats to various MV-HSV constructs and MV-Schwarz control virus at day 28, day 42, and day 49 after receiving two doses of a given construct. FIG. 7D shows serum neutralizing antibody (SNA) responses in cotton rats to various MV-HSV constructs and MV-Schwarz control virus at day 28, day 42, and day 49 after receiving two doses of a given construct.

FIG. 8A shows gB antibody responses in cotton rats to various MV-HSV constructs and MV-Schwarz control virus at day 28, day 42, and day 49 after receiving two doses of constructs MV-HSV constructs T-15 and T-16 with controls. FIG. 8B shows gC antibody responses in cotton rats to various MV-HSV constructs and MV-Schwarz control virus at day 28, day 42, and day 49 after receiving two doses of constructs MV-HSV constructs T-15 and T-16 with controls. FIG. 8C shows gD antibody responses in cotton rats to various MV-HSV constructs and MV-Schwarz control virus at day 28, day 42, and day 49 after receiving two doses of constructs MV-HSV constructs T-15 and T-16 with controls. FIG. 8D shows serum neutralizing antibody (SNA) responses in cotton rats to various MV-HSV constructs and MV-Schwarz control virus at day 28, day 42, and day 49 after receiving two doses of constructs MV-HSV constructs T-15 and T-16 with controls.

FIG. 9 shows a dot plot of vaginal viral load in cotton rats after treatment with MV-HSV constructs T-15 or T-16, gD protein/ML-A+alhydrogel, or MV-Schwarz, followed by viral challenge with HSV-2.

### DETAILED DESCRIPTION OF THE INVENTION

### Definitions and Abbreviations

As used throughout the specification and appended claims, the following abbreviations apply:
ATU additional transcriptional unit
bp base pairs
BSA bovine serum albumin
CDS coding sequence
CMV human cytomegalovirus immediate early enhancer and promoter
ELISA enzyme-linked immunosorbent assay
ER endoplasmic reticulum
GE gene end
GS gene start
HSV Herpes Simplex Virus
IM intra-muscular
MOI multiplicity of infection
MV measles virus
MV-HSV measles virus carrying one or more HSV proteins in its genome
nt nucleotide(s)
ORF open reading frame
PBS phosphate buffered saline
PFU plaque forming units
SNA serum neutralizing antibody(ies)
WT wild-type

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the term “including” as well as other forms, such as “include,”

“includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoints and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

### Attenuated Measles Virus

Measles virus (MV) is a non-segmented single-stranded, negative-sense enveloped RNA virus of the genus Morbilivirus within the family of Paramyxoviridae. Measles virus was isolated in 1954 (Enders, J. F., and T. C. Peebles. 1954. Propagation in tissue cultures of cytopathogenic agents from patients with measles. Proc. Soc. Exp. Biol. Med. 86:277-286), and live attenuated measles strains were derived from this virus to provide vaccines. Measles vaccines from live attenuated measles virus have been administered to hundreds of millions of children since 1963 and are well-known to be safe and efficacious in preventing measles infection. It is produced on a large scale in many countries and is distributed at low cost.

The disclosure describes attenuated recombinant measles virus particles that stably express one or more protein antigens of HSV (e.g., gD, gB, gC, and/or UL19). The disclosure also describes nucleic acid constructs which comprise an isolated cDNA encoding a full-length, infectious, attenuated antigenomic (+) RNA strand of a measles virus (MV) and at least one HSV glycoprotein (e.g., gD, gB, gC and/or UL19), such that a rescued MV comprises the at least one HSV protein in its genome.

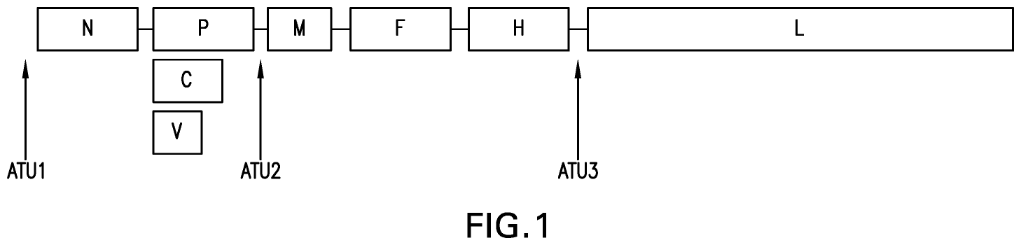

The non-segmented genome of measles virus (MV) has an anti-message polarity which results in a genomic RNA which is not translated in vivo or in vitro and is not infectious when purified. Transcription and replication of measles virus do not involve the nucleus of the infected cells but rather take place in the cytoplasm of infected cells. The genome of the measles virus comprises genes encoding six major structural proteins from the six genes (designated N, P, M, F, H and L) and an additional two non-structural proteins from the P gene (C and V). The gene order is the following: 3′, N, P (including C and V), M, F, H, and L (the L gene encoding for the large polymerase protein at the 5′ end (see schematic diagram of FIG. 1). The MV genome further comprises non-coding regions in the intergenic region M/F; this non-coding region contains approximately 1000 nucleotides of untranslated RNA. The MV genes respectively encode the proteins of the nucleocapsid of the virus, i.e., the nucleoprotein (N), the phosphoprotein (P), and the large protein (L) which assemble around the genome RNA to provide the nucleocapsid. The other genes encode the proteins of viral envelope including the hemagglutinin (H), the fusion (F) and the matrix (M) proteins.

In some embodiments, the MV used is an attenuated strain. As used herein, an “attenuated strain” of measles virus is a strain that is avirulent or less virulent than the parent strain in the same host, while maintaining immunogenicity and optionally adjuvanticity when administered to a host, i.e., preserving immunodominant T and B cell epitopes and possibly the adjuvanticity such as the induction of T cell costimulatory proteins or the cytokine IL-12.

An attenuated strain of a measles virus accordingly refers to a strain which has been serially passaged on selected cells and, possibly, adapted to other cells to produce seed strains suitable for the preparation of vaccine strains, harboring a stable genome which would not allow reversion to pathogenicity nor integration in host chromosomes. Particular strains of attenuated MV that can be used are the Schwarz strain, the Zagreb strain, the AIK-C strain and the Moraten strain. In specific embodiments, the attenuated strain of measles virus in any one of the embodiments or aspects herein is the Schwarz strain, the Zagreb strain, the AIK-C strain or the Moraten strain

In some embodiments of the invention, the vector for the rescue of a recombinant measles virus comprising the isolated nucleic acid molecule disclosed herein comprises a heterologous promoter sequence. Exemplary heterologous promoters include the CMV promoter sequence. In some embodiments, the vector is pBluescript KS (+) (GenBank X52331.1; SEQ ID NO: 1). In some embodiments, the vector is pBluescript II KS (+) (Agilent, Santa Clara, CA, United States, Cat. #212207, GenBank X52327.1; SEQ ID NO: 2). In some embodiments, the vector includes a T7 promoter sequence, a T7 terminator sequence, and a hammerhead ribozyme sequence. An exemplary sequence is that of plasmid pTM-MVSchw (SEQ ID NO: 3; see WO2004000876A1). The plasmid pTM-MVSchw is a Bluescript plasmid that comprises the polynucleotide coding for the full-length measles virus (+) RNA strand of the Schwarz strain placed under the control of the promoter of the T7 RNA polymerase.

In embodiments described herein, the MV-HSV cDNA includes HSV proteins inserted into an additional transcriptional unit (ATU). The term “additional transcriptional unit” or “ATU” in relation to the MV genome refers to an intergenic region of the MV genome having cis-acting 3′ and 5′ untranslated regions (UTRs) of the genes, which are composed of the non-coding sequences (NCS) and of conserved gene end (GE) and gene start (GS) signals necessary for the transcription of the immediately adjacent open reading frames. This “GE/GS stop-start signal” is comprised of a conserved GE sequence, a non-transcribed conserved trinucleotide sequence, and a conserved GS sequence (see Parks et al., J Virol. 2001 January;75 (2): 921-33). During transcription, each gene in a transcription unit of the MV genome is sequentially transcribed into mRNA by the viral RNA-dependent RNA polymerase that starts the transcription process at the GS sequence. At each gene junction, transcription is interrupted as a result of the disengagement of the RNA polymerase at the GE sequence. Re-initiation of transcription occurs at the subsequent GS sequence.

To enable the MV cDNA to act as a vector for the expression of one or more HSV heterologous genes, a multiple-cloning-site cassette having one or more HSV genes can be cloned into the intergenic region of the MV so as to maintain the MV non-coding sequences and the conserved gene end (GE) and gene start signals (GS) of the immediately adjacent open reading frames of the intergenic transcription unit in which it is inserted; see, e.g., the ATU region described for the EdB-tag vector in Radecke et al., 1997 Rev. Med. Virol. 7:49-63, and Wei et al., 2019 Biochem. Biophys. Res. Comm. 508:1221-1226. Following cloning, the resulting ATU contains an additional GE/GS stop-start signal suitable for the transcription of the one or more inserted heterologous HSV genes. When multiple heterologous HSV genes are inserted, each heterologous HSV gene may be separated by an “interstitial ATU,” which is an additional GE/GS stop-start signal that separates the heterologous HSV genes. In specific embodiments, the additional GE/GS stop-start signal is the same as that found in an MV intergenic region. In specific embodiments, the GE/GS stop-start signal is a variant of that found in an MV intergenic region.

It is important in all cases that the ATU shall comply with the broader “rule of six” to allow for the expression of the one or more heterologous genes.

In this disclosure, locations of ATUs along the MV genome are numbered. ATU position 1 (ATU1) is in the MV leader sequence before the N gene. ATU position 2 (ATU2) is in the intergenic region between the P and M genes. ATU position 3 (ATU3) is in the intergenic region between the H and L genes. Insertion of heterologous transcription units ATU2 and ATU3 can be accomplished as disclosed herein or as elsewhere described in the literature, for example, in Combredet et al., 2003 J. Virol. 11546-11554.

In some embodiments, an ATU cDNA comprises a GS sequence for an N gene and a GE sequence, such as at ATU1 in MV, or suitable GS and GE variants that are capable of starting and ending transcription, respectively, in the MV. For example, in specific embodiments, the ATU cDNA comprises a GE/GS sequence (GE/GS stop-start signal) comprising CTTCTAGTGCACTTAGGATTCAA (SEQ ID NO: 83), wherein the GE sequence is CTTCTAGTGCA (SEQ ID NO: 84), the conserved trinucleotide sequence is CTT (SEQ ID NO: 85), and the GS sequence is AGGATTCAA (SEQ ID NO: 86). In some embodiments, the ATU cDNA comprises the GE of an N gene and the GS of a P gene. In some embodiments, the ATU cDNA comprises GTTATAAAAAACTTAGGAACCAGGTCCACAC (ATU upstream motif; SEQ ID NO: 87), wherein the GE sequence is GTTATAAAAAA (GE of N gene; SEQ ID NO: 88), the conserved trinucleotide sequence is CTT (SEQ ID NO: 85), and the GS sequence is AGGAACCAGGTCCACAC (GS of P gene; SEQ ID NO: 89). In some embodiments, the ATU cDNA comprises the GE of a P gene and the GS of a M gene. In some embodiments, the ATU cDNA comprises ATTATAAAAAACTTAGGAGCAAAGTGATTGC (ATU downstream motif; SEQ ID NO: 90), wherein the GE sequence is ATTATAAAAAA (GE of P gene; SEQ ID NO: 91), the conserved trinucleotide sequence is GTT (SEQ ID NO: 85), and the GS sequence is AGGAGCAAAGTGATTGC (GS of M gene; SEQ ID NO: 92). In some embodiments, the ATU cDNA comprises a GE and GS sequence of the same gene combined with the conserved trinucleotide sequence. For example, in specific embodiments, the ATU cDNA comprises the GE and the GS of the P gene combined with a conserved trinucleotide sequence, i.e., ATTATAAAAAACTTAGGAACCAGGTCCACAC (ATUa; SEQ ID NO: 93). In some embodiments, the ATU cDNA comprises a hybrid GS sequence that combines portions of sequences from different MV intergenic regions. In some embodiments, the ATU cDNA comprises a hybrid GS sequence that is a combination of a GS sequence of an MV P gene and a GS sequence of an MV M gene, e.g., AGGAGCAAAGTCCACAC (SEQ ID NO: 94). In some embodiments, the ATU cDNA comprises a hybrid GS sequence combined with a GE from an N gene, e.g., GTTATAAAAAACTTAGGAGCAAAGTCCACAC (ATUb; SEQ ID NO: 100). In some embodiments, the hybrid GS sequence may be a consensus GS sequence, e.g., AGGATCCAAGAGCATAC (SEQ ID NO: 96). In some embodiments, the ATU cDNA comprises a hybrid GS sequence and a GE from an N gene, e.g., GTTATAAAAAACTTAGGATCCAAGAGCATAC (SEQ ID NO: 97).

ATU sequence that flanks the GE/GS sequence may be part or all of an intergenic region of an MV strain (e.g., the N-P, P-M, or H-L intergenic region of the Schwarz, Zagreb, AIK-C, Moraten, or Rubeovax MV strain) that is duplicated in a different intergenic region of the MV (see FIG. 1).

In some embodiments of the MV described herein, the heterologous HSV gene may be preceded by a Kozak sequence. The term “Kozak sequence” refers to a nucleic acid motif that acts as a protein translation initiation site for the heterologous gene or genes and includes the ATG initiation codon. In some embodiments, the Kozak sequence in a cDNA may be the sequence GCCGCCATG (SEQ ID NO: 98) or the sequence GCCACCATG (SEQ ID NO: 99).

Complementary DNA (cDNA) encoding MV-HSV as described herein complies with the “rule of six” which is required in order to express infectious viral particles. The term “rule of six” as used herein refers the fact that the total number of nucleotides present in the MV cDNA is a multiple of six. If the rule of six is not followed when adding heterologous genes, then replication of the MV genome RNA is inhibited (see Fields BN et al. (ed.). Fields Virology. 3^{rd} ed. Vol. 1. Raven Press; 1996 at p. 1197).

In some embodiments of the isolated nucleic acid molecules described herein, the HSV protein ORFs (the one or more HSV cDNAs encoding an HSV protein) are separated by a self-cleaving 2A peptide so that multiple separate peptides can be generated from a single ORF. The term “2A peptide”, “self-cleaving 2A peptide” or “2A self-cleaving peptide” refers to viral oligopeptides that are 18-22 amino acids in length and mediate cleavage of different polypeptides encoded by polycistronic mRNA during translation in eukaryotic cells. Coding sequences (CDS) for 2A peptides can be inserted between coding sequences for two polypeptides, and ribozyme skipping of the formation of glycyl-prolyl peptide bond at the C-terminus results in separation of the two polypeptides flanking the 2A peptide coding sequence (see Liu et al., Sci Rep. 2017 May 19;7(1):2193). A 2A peptide may be derived from various viruses, including but not limited to: T2A (thosea asigna virus 2A; SEQ ID NO: 4,GSGEGRGSLLTCGDVEENPGP); P2A (porcine teschovirus-1 2A; SEQ ID NO: 5,GSGATNFSLLKQAGDVEENPGP); E2A (equine rhinitis A virus; SEQ ID NO: 6,GSGQCTNYALLKLAGDVESNPGP); and foot-and-mouth disease virus (F2A; SEQ ID NO: 7,GSGVKQTLNFDLLKLAGDVESNPGP). In some embodiments, the GSG sequence at the N-terminal residues 1-3 can be removed, although this can decrease cleavage efficiency: T2A-SEQ ID NO: 8, EGRGSLLTCGDVEENPGP; P2A-SEQ ID NO: 9,

ATNFSLLKQAGDVEENPGP; E2A-SEQ ID NO: QCTNYALLKLAGDVESNPGP; F2A-SEQ ID NO: 10, VKQTLNFDLLKLAGDVESNPGP.

In some embodiments of the isolated nucleic acid molecules described herein, a furin cleavage sequence may be positioned between HSV antigen ORFs instead of a 2A peptide. In such embodiments, a peptide sequence is recognized by a furin enzyme in a cell and cleaved, allowing separation of polypeptides in the cell. Furin cleavage sequences are traditionally described by the consensus sequence RXRR (SEQ ID NO: 12) or RXKR (SEQ ID NO: 13), wherein X is any amino acid. In some embodiments, the furin-cleavage sequence may be SEQ ID NO: 14 (RGRR), SEQ ID NO: 15 (RARR), SEQ ID NO: 16 (RLRR), SEQ ID NO: 17 (RMRR), SEQ ID NO: 18 (RFRR), SEQ ID NO: 19 (RWRR), SEQ ID NO: 20 (RKRR), SEQ ID NO: 21(RQRR), SEQ ID NO: 22 (RERR), SEQ ID NO: 23 (RSRR), SEQ ID NO: 24 (RPRR), SEQ ID NO: 25 (RVRR), SEQ ID NO: 26 (RIRR), SEQ ID NO: 27 (RCRR), SEQ ID NO: 28 (RYRR), SEQ ID NO: 29 (RHRR), SEQ ID NO: 30 (RRRR), SEQ ID NO: 31 (RNRR), SEQ ID NO: 32(RDRR), SEQ ID NO: 33 (RTRR), SEQ ID NO: 34 (RGKR), SEQ ID NO: 35 (RAKR), SEQ ID NO: 36 (RLKR), SEQ ID NO: 37 (RMKR), SEQ ID NO: 38 (RFKR), SEQ ID NO: 39 (RWKR), SEQ ID NO: 40 (RKKR), SEQ ID NO: 41 (RQKR), SEQ ID NO: 42 (REKR), SEQ ID NO: 43(RSKR), SEQ ID NO: 44 (RPKR), SEQ ID NO: 45 (RVKR), SEQ ID NO: 46 (RIKR), SEQ ID NO: 47 (RCKR), SEQ ID NO: 48 (RYKR), SEQ ID NO: 49 (RHKR), SEQ ID NO: 50 (RRKR), SEQ ID NO: 51 (RNKR), SEQ ID NO: 52 (RDKR), or SEQ ID NO: 53 (RTKR).

In some embodiments of the isolated nucleic acid molecules described herein, a furin cleavage sequence is used in combination with a 2A peptide to ensure that no additional 2A peptide sequence remains after self-cleavage by the 2A peptide. The furin cleavage sequence is adjacent to the 2A peptide, between an antigen and a 2A peptide sequence (see Fang et al., Nat Biotechnol. 2005 May;23(5):584-90; and WO2015054639A1, each of which is incorporated herein by reference). In some embodiments, the GSG linker may be removed (see Chng et al., Mabs. 2015;7 (2): 403-12, incorporated by reference herein).

Various combinations of ATUs, GE/GS sequence, 2A peptides, and 2A peptides with furin cleavage sequences are contemplated for the isolated nucleic acid molecules described herein. In some embodiments of the isolated nucleic acid molecules described herein, a single HSV protein ORF encoding an HSV protein (e.g., HSV-2 gC, gBwt, gBmut, gBwtdel25, gD, or UL19, see SEQ ID NOs: 57, 55, 58, 64, 56, and 65) is flanked by an ATU upstream motif (e.g., SEQ ID NO: 87) and an ATU downstream motif (e.g., SEQ ID NO: 90). For example, cDNA encoding HSV-2 gC, gBwt, gBmut, gBwtdel25, or gD (see SEQ ID NOs: 57, 55, 58, 64, 56, and 65) may be positioned at ATU2 (e.g., gC, see SEQ ID NO: 59) or at ATU3 (e.g., gC, gBwt, gBmut, gD, or UL19, see SEQ ID NOs: 60-63, and 66).

In some embodiments, two HSV protein coding sequences (e.g., gC, gBwt, gBmut, gBwtdel25, gD, or UL19, see SEQ ID NOs: 57, 55, 58, 64, 56, and 65) flanked by an ATU upstream motif (e.g., SEQ ID NO: 87) and an ATU downstream motif (e.g., SEQ ID NO: 90) may be separated by an ATUa motif (SEQ ID NO: 93) or ATUb motif (SEQ ID NO: 95). In some embodiments, the two HSV protein coding sequences may be located at ATU2 or at ATU3 (e.g., SEQ ID NOs: 66, 68-70). In some embodiments, the two HSV protein coding sequences may be separated by a 2A peptide coding sequence (SEQ ID NOs: 4-11). For example, see SEQ ID NO: 67.

In some embodiments, three HSV protein coding sequences (e.g., gC, gBwt, gBmut, gBwtdel25, or gD, see SEQ ID NOs: 57, 55, 58, 64, 56, and 65) flanked by an ATU upstream motif (e.g., SEQ ID NO: 87) and an ATU downstream motif (e.g., SEQ ID NO: 90) may be separated by an ATUa motif (SEQ ID NO: 93), an ATUb motif (SEQ ID NO: 95), a 2A peptide motif (SEQ ID NOs: 4-11), a furin cleavage site (SEQ ID NOs: 12-53) and a 2A peptide motif (SEQ ID NOs: 4-11), and combinations thereof. Such coding sequences may be located at ATU2or at ATU3. For example, see SEQ ID NOs: 71-82.

### Nucleic Acids and Proteins

In some embodiments, the inventions disclosed herein refer to isolated cDNA encoding MV-HSV.

As used herein, the term “operably linked” refers to a functional relationship between two or more nucleic acid sequences. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the example of a secretory leader, in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “ORF” or “open reading frame” refers to a coding sequence of a gene that begins with the start codon, continues with the amino acid codons, and ends at a termination codon. A “gene” includes an ORF and includes sequences upstream of the start codon and downstream of the stop codon that may be useful for transcribing the ORF.

As used herein, the term “complementary DNA” or “cDNA” refers to a deoxyribonucleic acid (DNA) molecule obtained by reverse transcription of a ribonucleic acid (RNA) molecule, such as an mRNA molecule. The term “cDNA” refers to the fact that originally said molecule is obtained by reverse transcription of the full length genomic (−) RNA strand of the genome of viral particles of the measles virus. This should not be viewed as a limitation for the methods used for its preparation. Purified nucleic acids, including DNA are thus encompassed within the term cDNA.

As used herein, the term “isolated” used in the context of polypeptides or polynucleotides refers to polypeptides or polynucleotides that are at least partially free of other biological molecules from the cells or cell cultures in which they are produced. Such biological molecules include other nucleic acids, proteins, lipids, carbohydrates, or other material such as cellular debris and growth medium. It may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the polypeptides or polynucleotides.

In some embodiments of the MV-HSV constructs described herein, conservative amino acid substitutions may be used for the sequence of the encoded HSV antigens. As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In some embodiments of the HSV antigens encoded and expressed by the measles virus vector of the invention, the HSV antigens may have up to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more conservative amino acid substitutions. In some embodiments, the measles vector polypeptides may have up to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more conservative amino acid substitutions.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to the degree of sequence relatedness between two sequences of polynucleotide or polypeptide molecules as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods.

The term “percent identity” or “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, accounting for the number of gaps and the length of each gap, which needs to be introduced for optimal alignment of the two sequences. The comparison of sequences and determination of percent identity between two sequences can be accomplished using a mathematical algorithm in an alignment tool (e.g. the Needleman-Wunsch algorithm in an online tool).

As used herein, the term “global alignment” refers to an alignment of residues between two amino acid or nucleic acid sequences along their entire length, introducing gaps as necessary if the two sequences do not have the same length, to achieve a maximum percent identity. A global alignment can be created using the global alignment tool “Needle” from the online European Molecular Biology Open Software Suite (EMBOSS) (see www.ebi.ac.uk/Tools/psa/emboss_needle/) or the global alignment tool “BLAST® »>Global Alignment” from the National Center for Biotechnology Information (NCBI) (see blast.ncbi.nlm.nih.gov/Blast.cgi?PROGRAM=blastn&PAGE_TYPE=BlastSearch&PROG DEF AULTS=on&BLAST_INIT=GlobalAln&BLAST_SPEC=GlobalAln&BLAST_PROGRAMS=bl astn). Both of these global alignment tools incorporate the Needleman-Wunsch algorithm (Needleman, S.B. & Wunsch, C.D. (1970) “A general method applicable to the search for similarities in the amino acid sequences of two proteins.” J. Mol. Biol. 48:443-453). In a preferred embodiment, a global alignment of nucleotide sequences using BLAST Global Alignment uses the following default parameters: match score=2; mismatch score =−3; Gap Cost Existence score =5; Gap Cost Extension Score =2. In a preferred embodiment, a global alignment of protein sequences using BLAST Global Alignment uses the following default parameters: Gap Cost Existence =11; Gap Cost Extension =1.

In some embodiments, codons encoding amino acid sequences may be substituted using wobble degenerate codons. As used herein, the term “wobble degenerate codon,” refers to a codon encoding a naturally occurring amino acid in either DNA or RNA. Wobble degenerate codons, when present in mRNA, are recognized by a natural tRNA anticodon through at least one non-Watson-Crick, or wobble base-pairing (e.g., A-C or G-U base-pairing). Watson-Crick base-pairing refers to either the G-C or A-U (RNA or DNA/RNA hybrid) or A-T (DNA) base-pairing. When used in the context of mRNA codon-tRNA anticodon base-pairing, Watson-Crick base-pairing means all codon-anticodon base-pairings are mediated through either G-C or A-U.

In some embodiments, the nucleic acids encoding the HSV proteins are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park CA), NovoPro Bioscience Inc. (Shanghai, China), and/or proprietary methods. In some embodiments, the sequence is optimized using optimization algorithms.

In some embodiments, a codon-optimized sequence shares less than 95% sequence identity, less than 90% sequence identity, less than 85% sequence identity, less than 80% sequence identity, or less than 75% sequence identity to a naturally-occurring or wild type sequence.

In some embodiments, a codon-optimized sequence shares between 65% and 85% (e.g., between about 67% and about 85%, or between about 67% and about 80%) sequence identity to a naturally-occurring sequence or a wild-type sequence. In some embodiments, a codon-optimized sequence shares between 65% and 75%, or about 80% sequence identity to a naturally-occurring sequence or wild-type sequence.

In some embodiments, nucleic acid sequence may be codon optimized for expression in cells from a particular animal species, such as a human (e.g., Homo sapiens) or other primate (e.g., Macaca mulatta or Macaca fascicularis). This optimization allows increasing the efficiency of chimeric infectious particles production in cells without impacting the expressed protein(s).

### HSV Proteins

The genome of Herpes Simplex Viruses (HSV-1 and HSV-2) contains about 85 open reading frames, such that HSV can generate at least 85 unique proteins, 12 of which are glycoproteins.

HSV glycoprotein C (gC) is a glycoprotein involved in the viral attachment to host cells. HSV gC plays a role in host immune evasion (also called viral immunoevasion) by inhibiting the host complement cascade activation. An MV-HSV immunogenic composition ideally will induce gC specific antibodies that block gC/C3b binding. HSV gC has 96-99% amino acid identity among HSV-2 strains and 64-69% amino acid identity among HSV-1 strains. An example of an HSV glycoprotein C (F327A) is set forth in SEQ ID NO: 54.

Glycoprotein B (gB) is a viral glycoprotein involved in the viral cell activity of HSV and is required for the fusion of the HSV envelope with the cellular membrane. HSV gB has 98-99% amino acid identity among HSV-2 strains and 88-90% amino acid identity among HSV-1 strains. An example of an HSV glycoprotein B is set forth in SEQ ID NO: 55.

Glycoprotein D (gD) is an envelope glycoprotein that binds to cell surface receptors and/or is involved in cell attachment via poliovirus receptor-related protein and/or herpes virus entry mediator, facilitating virus entry. HSV gD has 98-99% amino acid identity among HSV-2 strains and 82-88% identity among HSV-1 strains. An example of an HSV glycoprotein D is set forth in SEQ ID NO: 56.

UL19 (also called VP5) is the major capsid protein for HSV. UL19 self-assembles to form an icosahedral capsid. The capsid is surrounded by a layer of proteinaceous material designated the tegument which, in turn, is enclosed in an envelope of host cell-derived lipids containing virus-encoded glycoproteins.

In some embodiments, an antigenic polypeptide suitable as an HSV protein includes gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain polypeptides or multichain polypeptides, such as antibodies or insulin, and may be associated or linked to each other. Most commonly, disulfide linkages are found in multichain polypeptides. The term “polypeptide” may also apply to amino acid polymers in which at least one amino acid residue is an artificial chemical analogue of a corresponding naturally occurring amino acid.

### Methods of Treatment

Provided herein are compositions (e.g., pharmaceutical compositions), methods, kits and reagents for prevention and/or treatment of HSV viral infection in humans and other mammals. MV-HSV immunogenic compositions can be used as therapeutic or prophylactic agents. They may be used in medicine to prevent and/or treat infectious disease. In exemplary aspects, the MV-HSV immunogenic compositions of the present disclosure are used to provide prophylactic protection from HSV virus infection. Prophylactic protection from HSV virus can be achieved following administration of an MV-HSV immunogenic compositions of the present disclosure. Immunogenic compositions can be administered once, twice, three times, four times or more. It is also envisioned that immunogenic compositions can be administered to an infected individual to achieve a therapeutic response. Dosing may need to be adjusted accordingly.

In some embodiments, the HSV immunogenic compositions of the present disclosure can be used as a method of preventing an HSV virus infection in a subject, the method comprising administering to the subject at least one MV-HSV immunogenic compositions as provided herein. In some embodiments, the MV-HSV immunogenic compositions of the present disclosure can be used as a method of treating an HSV virus infection in a subject, the method comprising administering to said subject at least one MV-HSV immunogenic compositions as provided herein. In some embodiments, the MV-HSV immunogenic compositions of the present disclosure can be used as a method of reducing an incidence of HSV virus infection in a subject, the method comprising administering to said subject at least one MV-HSV immunogenic compositions as provided herein. In some embodiments, the MV-HSV immunogenic compositions of the present disclosure can be used as a method of inhibiting spread of HSV virus from a first subject infected with HSV virus to a second subject not infected with HSV virus, the method comprising administering to at least one of said first subject and said second subject at least one MV-HSV immunogenic compositions as provided herein.

A method of eliciting an immune response in a subject against an HSV virus is provided in aspects of the invention. The method involves administering to the subject an HSV immunogenic composition described herein, thereby inducing in the subject an immune response specific to HSV virus antigenic polypeptide or an immunogenic fragment thereof.

A prophylactically effective dose is a therapeutically effective dose that prevents infection with the virus at a clinically acceptable level. In some embodiments the therapeutically effective dose is a dose listed in a package insert for the vaccine.

### Therapeutic and Prophylactic Compositions

Provided herein are compositions (e.g., pharmaceutical compositions), methods, kits and reagents for prevention, treatment or diagnosis of HSV infection in humans and other mammals, for example. MV-HSV immunogenic compositions can be used as therapeutic or prophylactic agents. They may be used in medicine to prevent and/or treat infectious disease. In some embodiments, immunogenic compositions in accordance with the present disclosure may be used for prevention and/or treatment of HSV infection.

MV-HSV immunogenic compositions may be administered prophylactically or therapeutically as part of an active immunization scheme to healthy individuals or early in infection during the incubation phase or during active infection after onset of symptoms. In some embodiments, the amount of immunogenic compositions of the present disclosure provided to a cell, a tissue or a subject may be an amount effective for immune prophylaxis.

MV-HSV immunogenic composition may be administered in one or more sequential doses. MV-HSV immunogenic compositions may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the same or different prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In some embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, MV-HSV immunogenic compositions may be administered intramuscularly or intradermally. In some embodiments, MV-HSV immunogenic compositions are administered intramuscularly.

MV-HSV immunogenic compositions may be utilized in various settings depending on the prevalence of the infection or the degree or level of unmet medical need. Immunogenic compositions have superior properties in that they produce much larger antibody titers and produce responses early than commercially available anti-viral agents/compositions.

Provided herein are pharmaceutical compositions including MV-HSV immunogenic compositions optionally in combination with one or more pharmaceutically acceptable excipients.

MV-HSV immunogenic compositions may be formulated or administered in combination with one or more pharmaceutically acceptable excipients. In some embodiments, immunogenic compositions comprise at least one additional active substances, such as, for example, a therapeutically active substance, a prophylactically active substance, or a combination of both. Immunogenic compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety). In some embodiments, MV-HSV immunogenic compositions are administered to humans, human patients or subjects.

Formulations of the MV-HSV immunogenic compositions described herein may be prepared by any method known or hereafter developed in the art of pharmacology. In general, such preparatory methods include the step of bringing the active ingredient (e.g., polypeptide or polynucleotide) into association with an excipient and/or one or more other accessory ingredients, and then, if necessary and/or desirable, dividing, shaping and/or packaging the product into a desired single-or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

### Modes of MV-HSV Immunogenic Composition Administration

MV-HSV immunogenic compositions may be administered by any route which results in a therapeutically or prophylactically effective outcome. These include, but are not limited, to intradermal, intramuscular, intranasal and/or subcutaneous administration. The present disclosure provides methods comprising administering immunogenic compositions to a subject in need thereof. The exact amount required will vary from subject to subject, depending on the species, age, and general condition of the subject, the severity of the disease, the particular composition, its mode of administration, its mode of activity, and the like. MV-HSV immunogenic compositions are typically formulated in dosage unit form for ease of administration and uniformity of dosage. It will be understood, however, that the total daily usage of immunogenic compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

An MV-HSV immunogenic pharmaceutical composition described herein can be formulated into a dosage form described herein, such as an intranasal, intratracheal, or injectable form (e.g., intravenous, intraocular, intravitreal, intramuscular, intradermal, intracardiac, intraperitoneal, intranasal and subcutaneous form).

### MV-HSV Virus Immunogenic Formulations and Methods of Use

Some aspects of the present disclosure provide formulations of the MV-HSV immunogenic composition, wherein the immunogenic composition is formulated in an effective amount to produce an antigen-specific immune response in a subject (e.g., production of antibodies specific to an HSV antigenic polypeptide). “An effective amount” is a dose of an immunogenic composition effective to produce an antigen-specific immune response. Also provided herein are methods of inducing an antigen-specific immune response in a subject.

In some embodiments, the antigen-specific immune response is characterized by measuring an anti-HSV antigenic polypeptide antibody titer produced in a subject administered an HSV immunogenic composition as provided herein. An antibody titer is a measurement of the level or concentration of antibodies within a sample from a subject, for example, antibodies that are specific to a particular antigen (e.g., an HSV antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous immunogenic composition was effective, and to identify any recent or prior infections. In accordance with the present disclosure, an antibody titer may be used to determine the strength of an immune response induced in a subject by an MV-HSV immunogenic composition.

The inventions of the disclosure are not limited in their application to the details of construction and the arrangement of components set forth in the following description or illustrated in the drawings. The inventions are capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

The following examples are meant to be illustrative and should not be construed as further limiting. The contents of the figures and all references, patents, and published patent applications cited throughout this application are expressly incorporated herein by reference.

### Example 1: Monovalent Recombinant Measles Viruses Carrying gB, gC, and gD

Monovalent recombinant measles viruses carrying various sequences of single HSV-2 antigens were generated and evaluated in vitro. The single antigens were gC (F327A) (SEQ ID NO: 57) at ATU2 or ATU3 of MV-Schwarz (SEQ ID NO: 3), mutated gB (gBmut; SEQ ID NO: 58) carrying additional mutations to reduce its toxicity, gB wild-type (gBwt; SEQ ID NO: 55), and gD wild-type (SEQ ID NO: 56). Table 2 below lists the monovalent MV-HSV constructs and their ATU locations.

TABLE 2
Monovalent MV-HSV constructs

| Vector name; | | |
|---|---|---|
| SEQ ID NO | Insert | ATU location |
| M-01 (SEQ ID | gC_ATU2 | ATU2 |
| NO: 59) | | |
| M-02 (SEQ ID | gC_ATU3 | ATU3 |
| NO: 60) | | |
| M-03 (SEQ ID | gBmut_ATU3 | ATU3 |
| NO: 61) | | |
| M-04 (SEQ ID | gD_ATU3 | ATU3 |
| NO: 62) | | |
| M-05 (SEQ ID | gBwt_ATU3 | ATU3 |
| NO: 63) | | |

### Viral Release Assay

Vero cells were seeded in T25 flasks at a cell density of 3.3E+04 cells per cm2 in a culture medium (Dulbecco's Modified Eagle Medium, DMEM GlutaMAX®, supplemented with 10% FCS) volume of 5 ml. Upon infection, culture medium was replaced with virus production-serum free medium (VP-SFM), supplemented with 4 mM L-glutamine and target virus (i.e., virus expressing heterologous HSV antigen) was added at an multiplicity of infection (MOI) of 0.01. As a control, additional cell culture flasks were infected with MV-Schwarz at the same MOI. Cell culture flasks were incubated at 32°° C., 5% CO2. Supernatants were harvested at day 3, 4, 5, 6 and 7 post-infection, centrifuged for 10 min at 900×g and 4° C., aliquoted and stored below −60° C. Subsequently, 5 ml VP-SFM+4 mM L-glutamine were added to the flasks containing the infected cells and the flasks were subjected to two freeze/thaw cycles. The detached frozen cells were then harvested and centrifuged for 10 min at 900 x g and 4° C. Supernatants of the lysed cells were then collected, aliquoted and stored below −60° C. The titer of the virus released from the cells (supernatant) as well as that of the cell associated viruses (i.e., virion formation within the cells; supernatant of lysed cells) were then determined by a TCID50 assay.

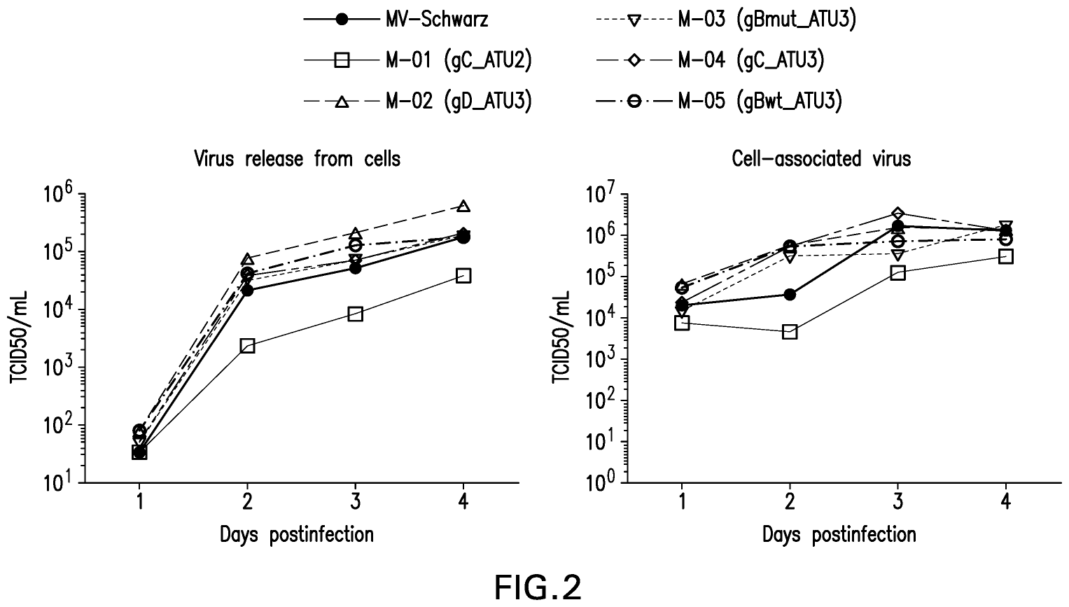

As shown in FIG. 2, MV vector expressing HSV-2 gC from position ATU2 (squares) demonstrated reduced viral release and reduced infectious viral particle formation within the cells compared to its counter vector with the insert at position ATU3 (diamonds). In addition, insertion of the wild-type sequence of HSV-2 gB at position ATU2 seemed to be too toxic for the virus as no virus with gBwt at position ATU2 could be rescued. Consequently, only measles vectors expressing the heterologous HSV-2 payloads at ATU3 were selected for further analysis in vivo.

### Example 2: Generation of Multivalent MV-HSV

Multivalent vectors were generated using the HSV-2 glycoproteins of Example 1. Two or three HSV-2 antigens were combined and separated by a) interstitial transcriptional units (interstitial ATUs); b) self-cleaving 2A peptides; or c) a combination thereof. Various inserts were designed in silico.

To improve the immunogenicity of the gB protein, a modified gB protein was constructed carrying a deletion of the last 25 amino acids of the cytoplasmic region—gBwtdel25 (SEQ ID NO: 64). This deletion removed the endoplasmic reticulum (ER) retention signal to allow increased surface expression in infected cells and increase the immunogenic potential of the MV.

Besides the HSV-2 antigens gB, gC and gD, a T-cell antigen UL19 (SEQ ID NO: 65; HSV-2 major capsid protein) was also selected as an additional target antigen and was combined with gBwt, gBmut or gBwtdel25. For each protein, a codon-optimization tool (www.novoprolabs.com/tools/codon-optimization) was used to generate a DNA stretch that encodes the desired protein with optimized codon usage for Homo sapiens. Long protein frames encoded in other frames as well as MV-editing sequences were removed by changing the 3rd nucleotide of a codon. The codon-optimized sequence was then reviewed to confirm that the codon usage of the alternative codon was of similar frequency to the originally suggested codon using a Homo sapiens codon frequency table (www.researchgate.net/figure/Homo-sapiens-codon-usage_tbl1_322560620).

The nucleotide sequences were chemically synthesized and cloned into the ATU3 position of MV-Schwarz. Finally, all bivalent and trivalent measles vectors as highlighted in Table 3 below were successfully rescued and subjected to further characterization in vitro.

TABLE 3
Bivalent and trivalent MV-HSV constructs

| Vector name; | Bivalent or | | Insert | ATU |
|---|---|---|---|---|
| SEQ ID NO | trivalent | Insert | size | location |
| B-06 (SEQ ID | bivalent | gD_ATUa_gC | ~2.8 kb | ATU3 |
| NO: 66) | | | | |
| B-07 (SEQ ID | bivalent | gD_P2A_gC | ~2.8 kb | ATU3 |
| NO: 67) | | | | |
| B-08 (SEQ ID | bivalent | gBwt_ATUa_UL19 | ~4.6 kb | ATU3 |
| NO: 68) | | | | |
| B-09 (SEQ ID | bivalent | gBmut_ATUa_UL19 | ~4.6 kb | ATU3 |
| NO: 69) | | | | |
| B-10 (SEQ ID | bivalent | gBwtdel25_ATUa_{—} | ~4.5 kb | ATU3 |
| NO: 70) | | UL19 | | |
| T-11 (SEQ ID | trivalent | gD_ATUa_gBmut_{—} | ~5.5 kb | ATU3 |
| NO: 71) | | ATUb_gC | | |
| T-12 (SEQ ID | trivalent | gD_P2A_gBmut_{—} | ~5.5 kb | ATU3 |
| NO: 72) | | T2A_gC | | |
| T-13 (SEQ ID | trivalent | gD_ATUa_gBmut_{—} | ~5.5 kb | ATU3 |
| NO: 73) | | P2A_gC | | |
| T-14 (SEQ ID | trivalent | gD_ATUa_gBwt_{—} | ~5.5 kb | ATU3 |
| NO: 74) | | P2A_gC | | |
| T-15 (SEQ ID | trivalent | gD_ATUa_gBwt_{—} | ~5.6 kb | ATU3 |
| NO: 75) | | ATUb_gC | | |
| T-16 (SEQ ID | trivalent | gD_ATUa_{—} | ~5.5 kb | ATU3 |
| NO: 76) | | gBwtdel25_ATUb_{—} | | |
| | | gC | | |

### Example 3: Antigen Expression in Bivalent and Trivalent Constructs

The expression of the heterologous recombinant antigens encoded by the various viruses was analyzed by an immunofocus staining assay using an alkaline phosphatase detection system (BCIP/NBT-plus substrate for ELISpot (Mabtech, Nacka Strand, SE; Cat. No.: 3650-10). Briefly, Vero cells were infected with various viruses of passage 1. Cells were fixed three days post-infection, cell membranes were permeabilized, and the heterologous antigen expression was detected using specific mouse monoclonal antibodies against gD (proprietary), gB (proprietary), gC (proprietary), and UL19 (Anti-HSV-1/2 ICP5 Major Capsid Protein Antibody (3B6); Santa Cruz Biotechnology, Dallas, TX, USA, Cat. No. sc-56989). To test for expression of measles proteins, cells were also stained for the measles virus H protein. Spots on the cell layer expressing the respective antigen exhibit a dark-blue coloring.

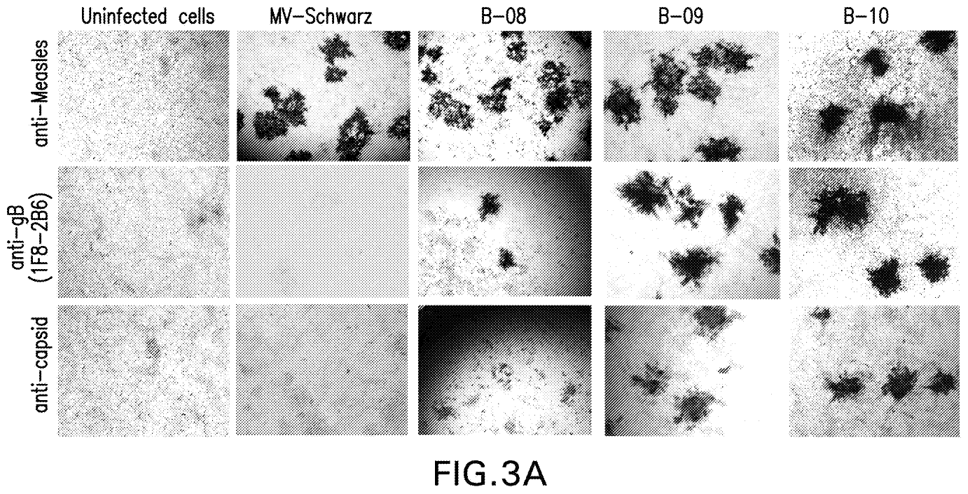

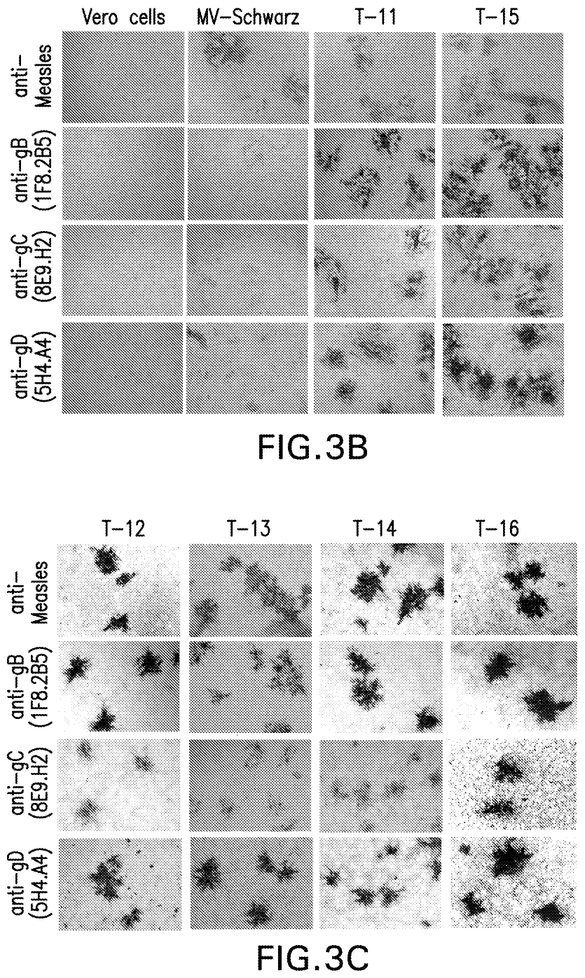

FIG. 3A shows the antigen expression of bivalent constructs, while FIGS. 3B and 3C show the expression of trivalent MV constructs. All viruses expressed the desired antigens. The trivalent vector expressing gD, gC (F327A) and gBwt (T-15) (see FIG. 3C), expressed all three antigens successfully. In contrast to T-15, two other trivalent viruses that carry a combination of an ATU and a 2A self-cleaving peptide (T-13 and T-14) showed impaired expression (see FIG. 3C). Surprisingly, the gC antigen downstream of the 2A self-cleaving peptide expressed less strongly than gD or gB in these constructs, as evidenced by very faint staining compared with gC expressed in other constructs.

The trivalent MV vector T-15 was also subjected to fluorescent-based immunostaining to allow for measles and HSV protein specific double staining (staining not shown in Figures). Vero cells infected with T-15, were fixed, permeabilized, and blocked with phosphate buffered saline (PBS)/0.05% Tween 20/1% bovine serum albumin (BSA). After blocking, measles and HSV protein specific double staining was performed using mouse MAbs for HSV gD (anti-gD 5H4A4), HSV gB (anti-gB 1F8.2B5), HSV gC (anti-gC 8E9.H2) and MV NP (Rabbit anti measles NP Ab, OriGene, AP55070SU-N) diluted in blocking solution. Measles NP expression was detected using AlexaFluor® 594 goat anti-rabbit secondary antibody (Life Technologies A11012), and heterologous antigen expression was detected using Alexa Fluor® 488 AffiniPure Goat Anti-Mouse IgG (H+L) (Jackson Laboratories; 115-545-003) secondary antibody. Images were captured using the ImageXpress® Pico Automated Cell Imagine (Molecular Devices, San Jose, CA, USA).

Because the HSV specific antibodies are derived from mouse, no triple staining could be performed. All cells infected with T-15 showed staining for both measles NP protein and HSV antigens, indicating all syncytia expressed the measles NP protein as well as the respective HSV antigen. There were no unstained or single stained syncytia detectable.

### Example 4: Growth Kinetics of Bivalent and Trivalent Constructs

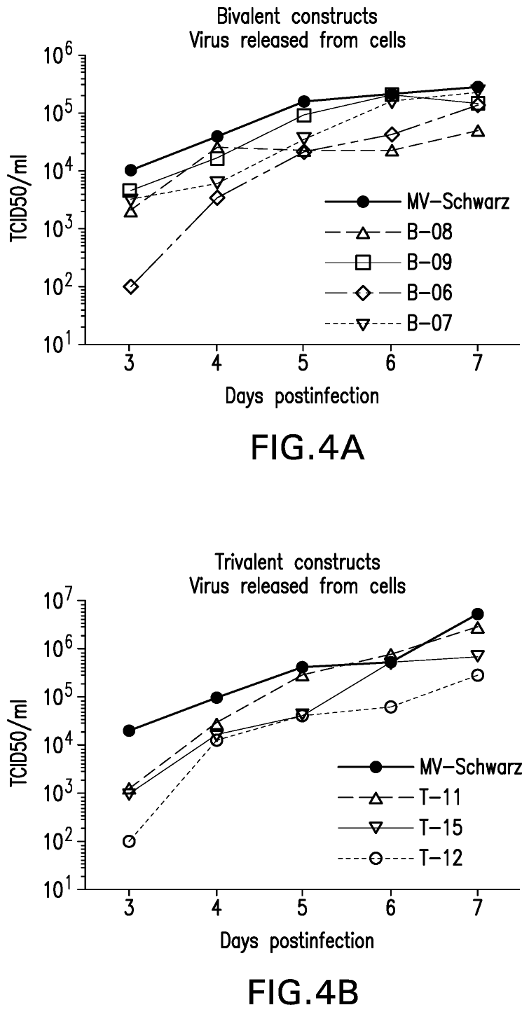

To assess the replication capacity of the various viruses, growth curve analysis of bivalent and trivalent MV expressing either two or three HSV-2 antigens was performed. Virus material of passage 1 was used for analysis. Briefly, Vero cells seeded in T-25 flasks were infected with a defined MOI of 0.01. As a control, additional cell culture flasks were infected with the parental MV-Schwarz at the same MOI. Supernatants were then collected at different time points and the titer of virus released from cells was determined by TCID50 assay. The results are shown in FIG. 4.

Most of the bivalent vectors exhibited a similar virus production as the parental Schwarz virus. Only the bivalent vector expressing a wild-type version of the gB protein was clearly impaired. Interestingly, two of the trivalent MV vectors in which the antigens were combined with two ATU sequences, T-011 and T-15, also showed a good virus release reaching high titers around days 6 or 7. Again, the virus with the gBwt protein (T-15) was more impaired than the virus with the mutant version indicating again the potential toxicity of the gB protein. Nevertheless, the T-15 vector was considered superior to T-011, as the monovalent MV vector expressing the mutated gB version exhibited a clearly reduced induction of antibodies compared to the vector expressing gB wild-type. These data indicate that the gB modifications may have affected the protein conformation. The replication of the third trivalent vector T-12 containing T2A peptides was clearly affected by the heterologous insert, showing a reduced virus production at all time points.

### Example 5: Trivalent Vector With Modified gB Protein

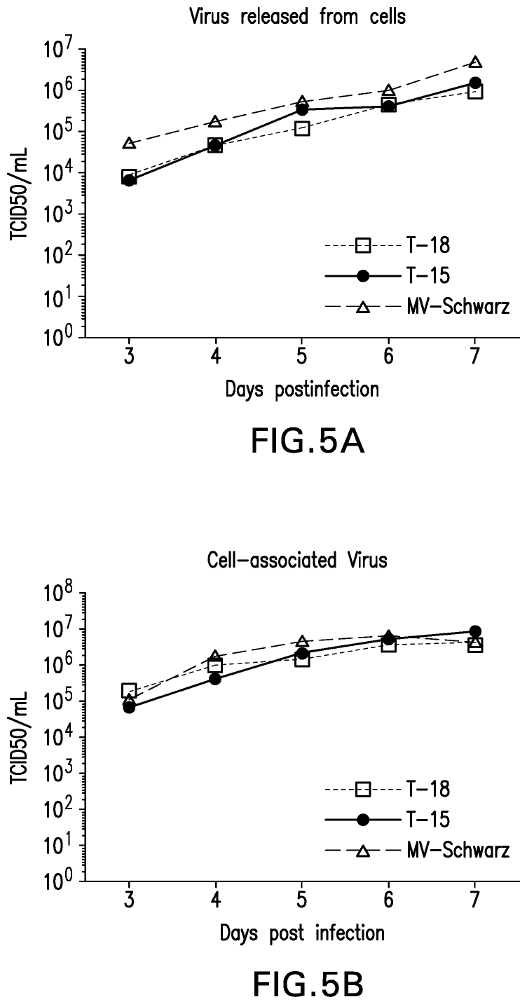

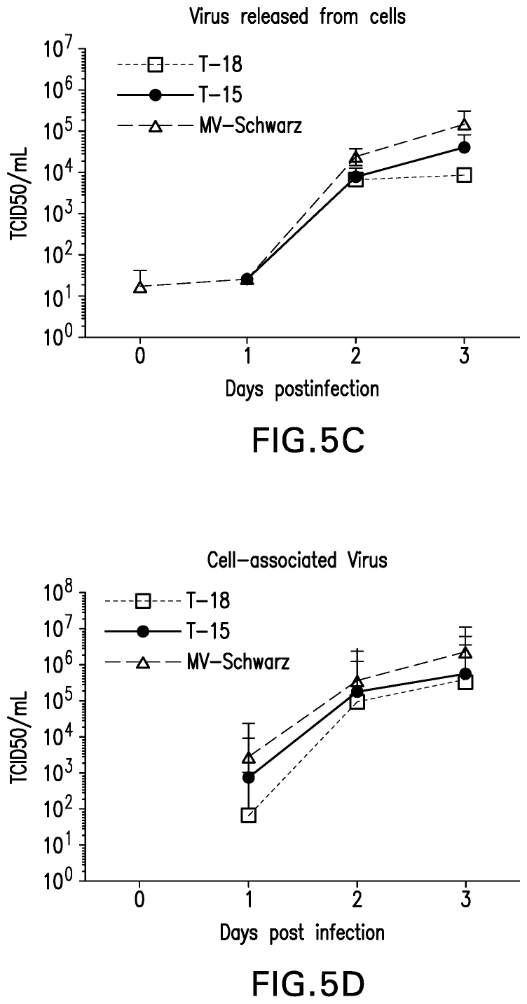

Another trivalent vector with a modified gB protein gBwtdel25 (T-18; MV-gD_ATU-gBwtdel25_ATU_gC) was generated and the growth behavior was compared to the trivalent MV vector T-15. Passage 2 material was used for analysis. Again, Vero cells in T25 flasks were infected with an MOI of 0.01 and supernatants as well as cell-associated viruses (supernatants from lysed cells) were analyzed at late (FIGS. 5A and 5B) as well as at early (FIGS. 5C and 5D) time points.

Interestingly, both viruses grew to high titers and were only slightly impaired in viral replication compared to the parental Schwarz strain. The delay in virus release of T-15 as seen in Example 4 above with Passage 1 material was clearly less pronounced. This outcome was confirmed in two independent experiments. Overall, the data suggested that both T-15 and T-18 were able to replicate well and grow to high titers and thus both were selected for efficacy studies in cotton rats.

### Example 6: Incorporation of HSV Antigens on Measles Virions

To assess whether measles virus can incorporate HSV antigens into their viral membrane, confocal microscopy was used to investigate the cell compartments in which the HSV glycoproteins of T-15 and T-16 localize.

Both viruses expressed the native forms of gD and gC but differed in the expression of gB; T-15 expressed the wild-type version of gB while T-16 expressed the 25 amino acid deletion variant of gB. Deletion of the last 25 aa of the gB cytoplasmic domain removes the ER retention signal and would potentially increase its surface expression in infected cells and enhance the antibody responses to the antigen in vivo. Deletion of the cytoplasmic domain changed the localization of the protein. Cells infected with T-16 gBwtdel25 showed more gBwtdel25 staining on the cell surface (data not shown). In addition, expression of the gD protein seemed to be slightly affected by the expression of gBwtdel25 (data not shown). Comparison of T-15 and T-16 staining revealed that gD of T-16 was more localized on the cell surface than within the intracellular compartments (data not shown). No difference was seen in the expression of glycoprotein gC which exhibited intracellular localization in both T-15 and T-16 (data not shown).

The concentrations of gB, gC and gD in the bulk drug substance were quantified by Simple Western, and the abundance of the HSV glycoprotein on measles virions were compared to those on HSV-2 virions, assuming all three antigens are associated with virions. As shown in Table 4 below, only low levels of HSV antigens were detected in T-15.

TABLE 4
Abundance of HSV-2 gB, gC, and gD in T-15 virions

| MV construct | gB/particle | gC/particle | gD/particle |
|---|---|---|---|
| or Control | % HSV-2 | % HSV-2 | % HSV-2 |
| T-15 | 8% | 2% | 8% |
| HSV-2 Control | 100% | 100% | 100% |

### Example 7: Genetic Stability of T-15 Construct During Passaging

The genetic stability of T-15 was tested in T-flasks. The virus was passaged five times. Infection of each passage was performed with a MOI of 0.01, and virus was propagated for 6 days at 32° C., before the supernatant was harvested.

Harvested supernatants of each passage were subjected to RNA extraction, subsequent cDNA synthesis amplification of the genomic insert using primers ATU3-PCR-1F1 (SEQ ID NO: 77) and ATU3-PCR-1R1 (SEQ ID NO: 78), and agarose gel electrophoresis to check for large-scale deletions in the T-15 HSV genomic insert. Passage 4 & 7 (p4 & p7) harvested supernatants were also subjected to Sanger sequencing of the insert.

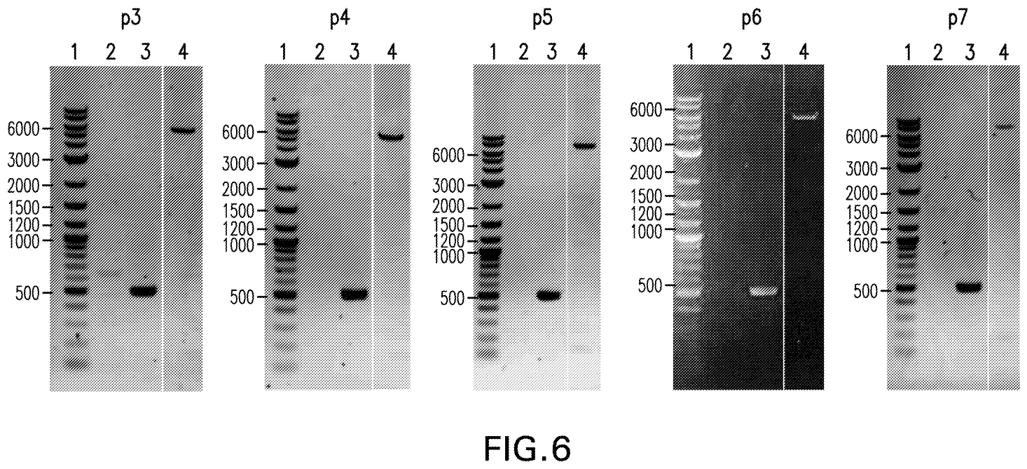

Amplicons of the genomic insert are shown in FIG. 6. Gel electrophoresis revealed that a PCR product of the anticipated size was readily detectable over the course of the virus passages. In addition, Sanger sequencing of the insert at Passage 4 and 7 confirmed the integrity of the insert. All these data together show that MV construct T-15 is genetically stable. Table 5 below lists the gel lanes, samples, and respective expected band size.

TABLE 5
Sample and expected band sizes for FIG. 6

| Lane | Sample | Expected band size (bp) |
|---|---|---|
| 1 | DNA marker | — |
| 2 | Neg. Control | — |
| 3 | MV-Schwarz p2 | 503 |
| 4 | T-15 p3-p7 | 6197 |

### Example 8: Bivalent and Trivalent MV Immunogenicity in Cotton Rats

Mice and guinea pigs are well established models to test and screen HSV-2 vaccines preclinically. However, these animal models are not permissive for measles virus infection. Cotton rats were selected as the preclinical small animal model because they are semi-permissive for measles virus infection and can be infected with HSV-2 following vaginal challenge. Like guinea pigs, cotton rats can exhibit lesion formation after HSV infection and can experience spontaneous recurrent vaginal disease after recovering from initial infection. Therefore, cotton rats can serve as a model for testing vaccine efficacy to prevent reactivation and recurrent disease.

### Measuring Antibody Titers and Viral Load

Antibody titers were measured using ELISA methodologies. Maxisorp plates were coated with recombinantly expressed gB, gC, or gD, and blocked with 3% milk in PBS-T. 4-fold serial serum dilutions were prepared starting from a 1:50 dilution in blocking buffer and transferred to assay plates. Binding was detected using species-specific horseradish peroxidase (HRP)-conjugated secondary antibodies. Interpolated titers were calculated using data from the dilutions for which the signal was directly above and below a predetermined threshold. This threshold was selected to match titers measured in this assay with titers previously measured in an alternate assay format. Serum samples that did not generate a signal that crossed the threshold value were reported as a titer of 25.

Viral load in cotton rats following HSV-2 infection was evaluated using plaque assays. Vero cells were seeded in 24-well plates and grown overnight. These cells were washed with serum free media immediately prior to infection. Vaginal swabs were placed in PBS at collection and were stored at 4° C. prior to analysis. These solutions were vortexed for 5-10 seconds, serially diluted in serum free culture media, and then transferred to the washed Vero cells. Plates were incubated for 1 hour at 37° C. and rocked every 15 minutes. After incubation, inoculum was aspirated, cells were overlaid with 0.75% methylcellulose in culture media containing 1.6% FBS, and plates were incubated at 37° C. for 3 days. Cells were then fixed and stained with crystal violet-glutaric dialdehyde, washed with water, and dried. Plaques were counted manually.

### MV Vector Administration

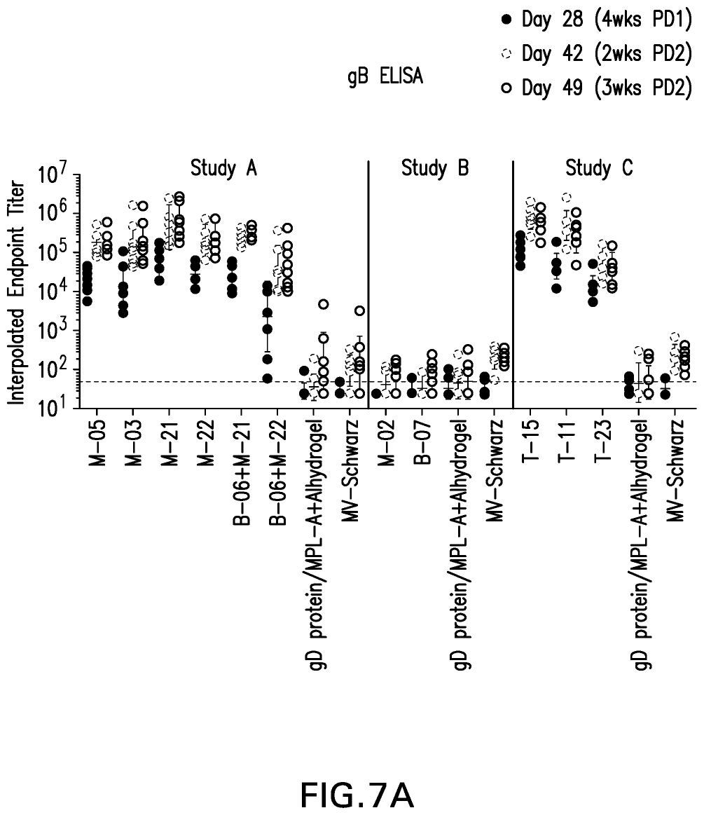

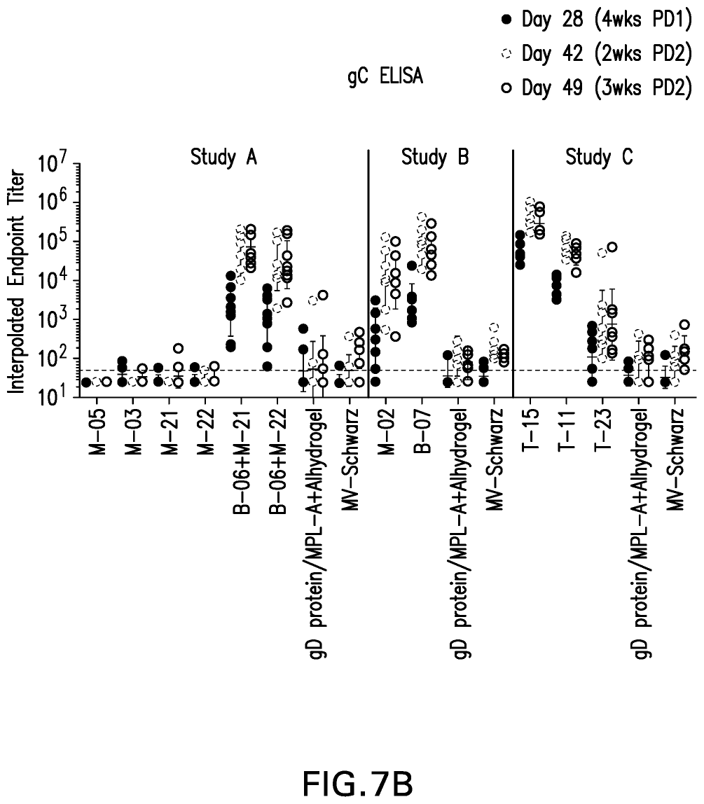

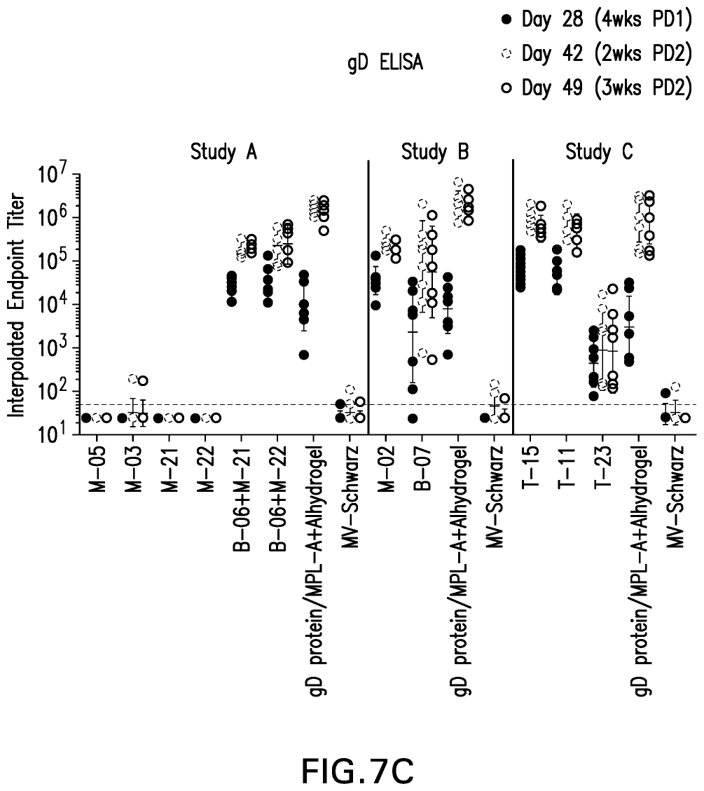

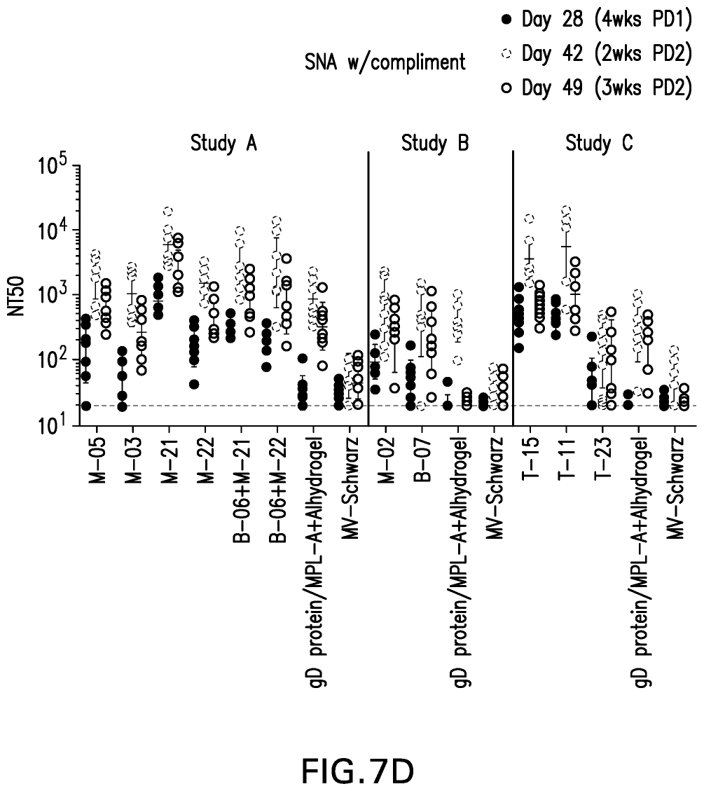

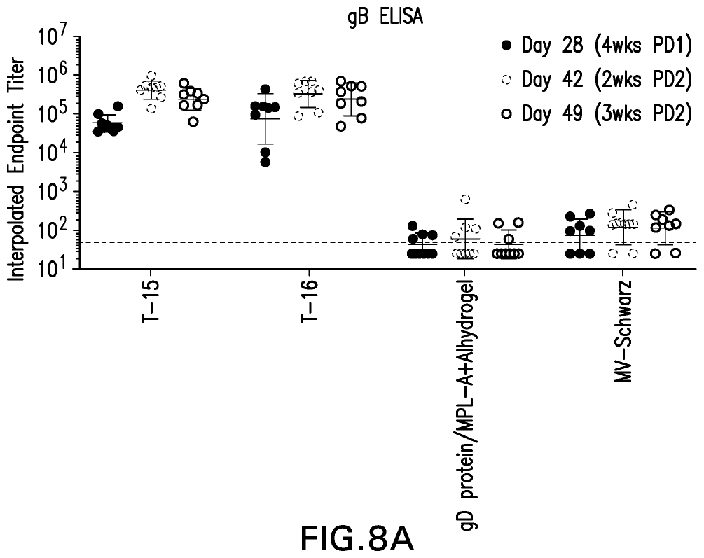

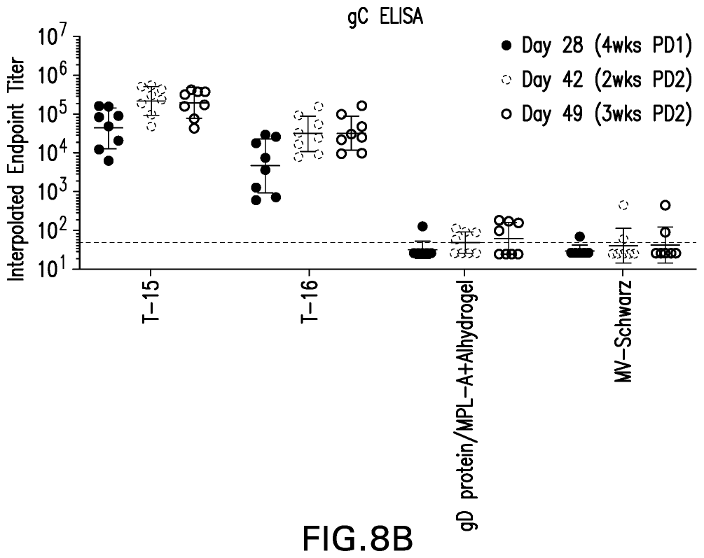

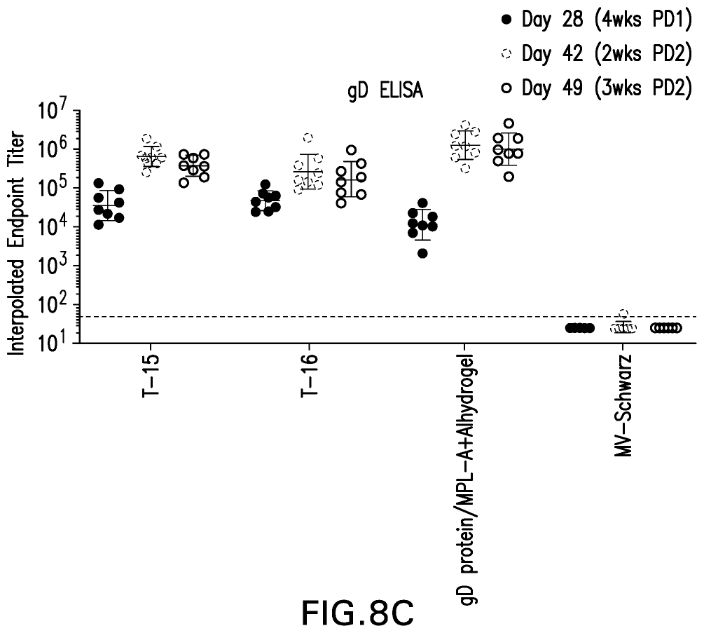

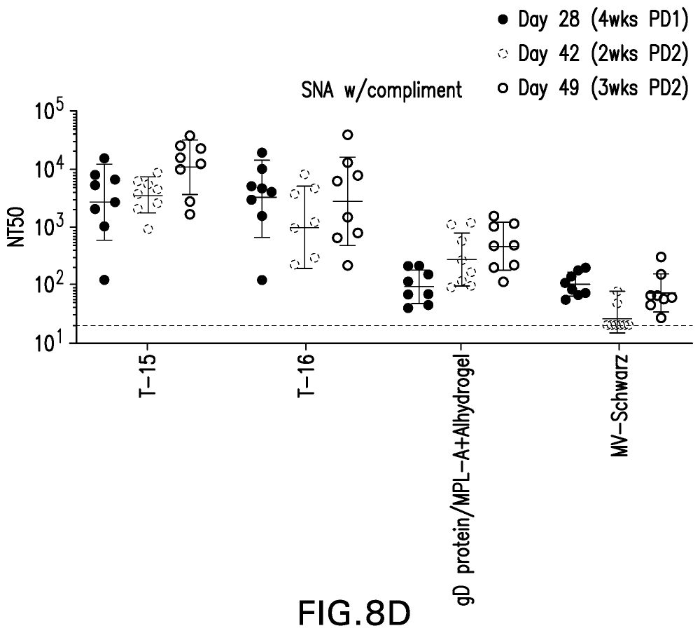

Mono-, di-, or trivalent antigen measles virus vectors were administered IM to cotton rats in two doses 28 days apart at 1×10⁵ TCID50. MV vectors were administered in 3 separate immunogenicity studies (Study A, Study B, Study C in FIGS. 7A-C and FIGS. 8A-8C). MV vectors tested were:
M-05 (insert gBwt_ATU3; SEQ ID NO: 63)
M-03 (insert gBmut_ATU3; SEQ ID NO: 61)
M-21 (insert gBwtdel25_ATU3; SEQ ID NO: 101)
B-06 (insert gD_ATUa gC_ATU3; SEQ ID NO: 66) administered in combination with M-21 (insert gBwtdel25_ATU3; SEQ ID NO: 101)
B-06 (insert gD_ATUa_gC_ATU3; SEQ ID NO: 66) administered in combination with M-22 (insert gBmutdel25_ATU3; SEQ ID NO: 102)
M-02 (insert gCwt_ATU3; SEQ ID NO: 60)
B-06 (insert gD_ATUa_gC_ATU3; SEQ ID NO: 66)
B-07 (insert gD_P2A_gC_ATU3; SEQ ID NO: 67)
T-15 (insert gD_ATUa_gBwt_ATUb_gC_ATU3; SEQ ID NO: 75)
T-11 (insert gD_ATUa_gBmut_ATUb_gC_ATU3; SEQ ID NO: 71)
T-23 (insert gD_P2A_gBmut_T2A_gC_ATU3; SEQ ID NO: 103)

For each immunogenicity study, sera were collected at four weeks post dose one (Day 28), and at two (Day 42) and three (Day 49) weeks post dose two. The gB, gC and gD specific IgG antibody titers were determined by ELISA using purified recombinant proteins as capture antigens substrates. Serum antibodies capable of neutralizing HSV-2 in the presence of exogenous complement also were quantified at the same time points.

### Results

As shown in FIG. 7A, gB antibody responses were only observed in MVs expressing the gB antigen, with the highest levels of response observed in the MVs T-15 and T-11. Similarly, the gC antibody specific responses were only observed in MVs expressing the gC antigen; again the trivalent viruses produced the highest antibody titers (FIG. 7B). The virus-induced gD antibody responses were equivalent to the responses generated by the positive control MV consisting of gD protein (5 mcg) in monophosphoryl lipid A (MPL-A)+Alhydrogel adjuvant (FIG. 7C). Most MVs showed serum neutralizing antibody (SNA) titers that were higher than the titers observed for the positive control vaccine gD/MPL-A+Alhydrogel (FIG. 7D). Collectively, these results demonstrate that the trivalent MVs TM-15 and T-11 induce robust antibody responses.

### Example 9: Trivalent MV Efficacy in Cotton Rats

To quantify the efficacy of the trivalent MVs T-15 and T-16, cotton rats were immunized using the same schedule and concentration that was employed in the immunogenicity studies. Three weeks after the second dose of the virus, the cotton rats were vaginally challenged with a lethal dose of HSV-2 strain MS. Survival and disease incidence were monitored for 28 days post challenge. Vaginal swabs were collected at 2 and 4 days post-challenge to quantify protection against acute viral shedding.

### Methods

Cotton rats were observed daily for 28 days post genital HSV-2 challenge. Time to clinical signs was defined as the first day a lesion was observed. For each group, the date was averaged, and the mean and standard error of the means calculated. If no clinical signs were observed for the 28 days no data is reported. Incidence was defined as the percentage of cotton rats in each group that showed at least one clinical sign. Mean survival time is the average of the day that each cotton rat succumbed to the challenge for each group (±standard error of the means). If the cotton rats survived the full 28 days no mean survival time is reported. Survival was defined as the percentage of cotton rats that survived to the end of the study.

### Results

Both MV T-15 and T-16 produced high levels of gB, gC, and gD specific antibody response (FIG. 8A-8C) and high levels of SNA titers (FIG. 8D) that were equivalent or higher than the positive control virus. Most of the trivalent-and positive control-vaccinated cotton rats were protected from primary genital disease compared to the cotton rats vaccinated with the measles virus Schwarz strain negative control (Table 6 below). This protection from primary disease translated to a 100% survival rate in these cotton rats compared to the 0% survival rate in the negative control group.

TABLE 6
Clinical signs in cotton rats treat with T-15, T-16

| | Mean Time | | | | Mean Survival | | | |
|---|---|---|---|---|---|---|---|---|
| | to Clinical | | | Incidence | Time ± SE | | | Survival |
| Group | Signs ± SE (Days) | | | (%) | (Days) | | | (%) |
| MV-gD_ATUa_gBwt_ATUb_gC | — | ± | — | 0 | — | ± | — | 100 |
| (T-15) | | | | | | | | |
| MV-gD_ATUa_gBwtdel25_ATUb_gC | 9.0 | ± | — | 13 | — | ± | — | 100 |
| (T-16) | | | | | | | | |
| gD protein/MPL-A + alhydrogel | 23.0 | ± | — | 14 | — | ± | — | 100 |
| MV-Schwarz | 10.6 | ± | 2.1 | 100 | 12.4 | ± | 1.9 | 0 |

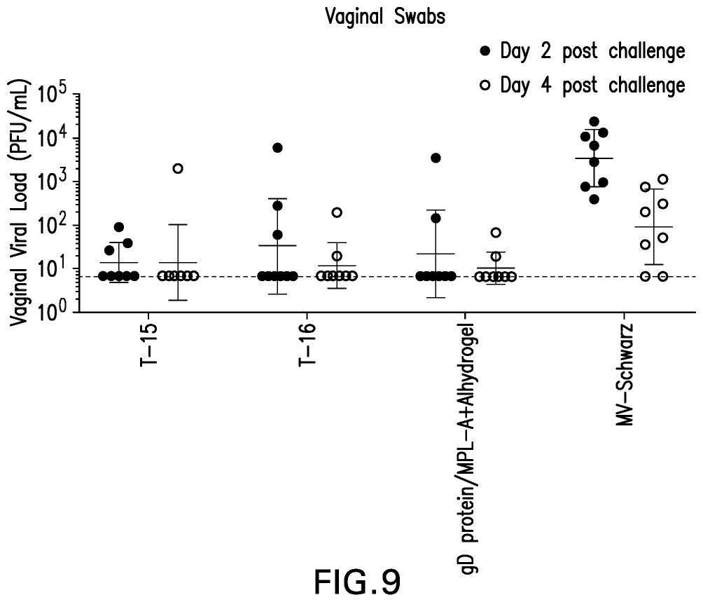

The vaginal swabs collected 2 days post-viral challenge were used to quantify viral shedding using a plaque assay. There was a 3-log reduction in viral titers for the trivalent and positive control vaccinated cotton rats compared to the negative control virus (See FIG. 9). Additionally, over half of the cotton rats in these groups showed undetectable levels of viral shedding. The effect of treatment was maintained at 4 days although the viral titers to the control virus were reduced by day 4 (FIG. 9). These immunogenicity and efficacy results indicate that both trivalent MV-HSV candidates provided protection against genital HSV-2 challenge that were as good as or better than the positive control virus.

Lengthy table referenced here
US20250312441A1-20251009-T00001
Please refer to the end of the specification for access instructions.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

LENGTHY TABLES
The patent application contains a lengthy table section. A copy of the table is available in electronic form from the USPTO web site (https://seqdata.uspto.gov/?pageRequest=docDetail&DocID=US20250312441A1). An electronic copy of the table will also be available from the USPTO upon request and payment of the fee set forth in 37 CFR 1.19(b)(3).

1. An isolated nucleic acid molecule comprising:
a) a cDNA encoding a full length, antigenomic (+) RNA strand of an attenuated strain of measles virus (MV-cDNA);
b) one or more cDNAs encoding an herpes simplex virus (HSV) protein (HSV cDNA) selected from the group consisting of: gC, gD, gB, UL19, and variants thereof;
c) an upstream additional transcriptional unit (ATU) cDNA operably linked to the HSV cDNA that is 5′ of the HSV cDNA (upstream ATU cDNA); and
d) a downstream ATU cDNA operably linked to the HSV cDNA that is 3′ of the HSV cDNA (downstream ATU cDNA);
wherein the upstream ATU cDNA, the HSV protein cDNA, and the downstream ATU cDNA are between P and M genes of the MV-cDNA at ATU2 or between H and L genes of the MV-cDNA at ATU3.

2. The isolated nucleic acid molecule of claim 1, wherein each of the one or more HSV cDNAs encodes an HSV protein sequence independently selected from the group consisting of: SEQ ID NO: 54 (HSV-2 gC, wild-type); SEQ ID NO: 57 (HSV-2 gC F327A);
SEQ ID NO: 55 (HSV-2 gB, wild-type); SEQ ID NO: 58 (HSV-2 gBmut); SEQ ID NO: 58.1 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19).

3. The isolated nucleic acid of claim 1, wherein the upstream ATU cDNA, the HSV cDNA, and the downstream ATU cDNA is at ATU2 or ATU3 in the MV-cDNA.

4. (canceled)

5. The isolated nucleic acid molecule of claim 1, wherein the upstream ATU cDNA sequence is set forth in SEQ ID NO: 87 or 90.

6. (canceled)

7. The isolated nucleic acid molecule of claim 1, comprising a sequence selected from the group consisting of SEQ ID NOs: 59-63 and 96-97.

8. An isolated nucleic acid molecule comprising:
a) a cDNA encoding a full length, antigenomic (+) RNA strand of an attenuated strain of measles virus (MV-cDNA);
b) a first cDNA encoding an herpes simplex virus (HSV) protein selected from the group consisting of: gC, D, gB, UL19, and variants thereof (first HSV cDNA);
c) a second HSV cDNA encoding an HSV protein selected from the group consisting of: gC, gD, gB, UL19, and variants thereof (second HSV cDNA), wherein the first and second HSV cDNAs do not have the same sequence;
d) an upstream additional transcriptional unit (ATU) cDNA that is 5′ of the first HSV cDNA (upstream ATU cDNA);
e) a downstream ATU cDNA that is 3′ of the second HSV cDNA (downstream ATU cDNA); and
f) an interstitial ATU cDNA between the first and second HSV cDNAs (interstitial ATU cDNA);
wherein the upstream ATU cDNA, the first and second HSV cDNAs, the interstitial ATU cDNA and the downstream ATU cDNA are operably linked; and
wherein the upstream ATU cDNA, the first and second HSV cDNAs, the interstitial ATU, and the downstream ATU cDNA are between P and M genes of the MV-cDNA at ATU2 or between H and L genes of the MV-cDNA at ATU3.

9. The isolated nucleic acid molecule of claim 8, wherein the first and second HSV cDNAs each encode an HSV protein sequence independently selected from the group consisting of SEQ ID NO: 54 (HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO: 58.1 (HSV-2 gBmutdel25); SEQ ID NO: 64 (HSV-2 gBwtdel25); SEQ ID NO: 56 (HSV-2 gD, wild-type);
and SEQ ID NO: 65 (HSV-2 UL19).

10. The isolated nucleic acid of claim 8, wherein the upstream ATU cDNA, the first and second HSV cDNAs, and the downstream ATU cDNA are at ATU2 or ATI3 in the MV-cDNA.

11. (canceled)

12. The isolated nucleic acid molecule of claim 8, wherein the upstream ATU cDNA sequence is set forth in SEQ ID NO: 87 or 90.

13. (canceled)

14. The isolated nucleic acid molecule of claim 8, wherein the interstitial ATU cDNA sequence is selected from the group consisting of SEQ ID NOs: 83, 87, 90, and 92.

15. The isolated nucleic acid molecule of claim 8, comprising a sequence selected from the group consisting of SEQ ID NOs: 66, 68, 69, and 70.

16. An isolated nucleic acid molecule comprising:
a) a cDNA encoding a full length, antigenomic (+) RNA strand of an attenuated strain of measles virus (MV-cDNA);
b) a first cDNA encoding an herpes simplex virus (HSV) protein (HSV cDNA) selected from the group consisting of: gC, gD, gB, UL19, and variants thereof (first HSV cDNA);
c) a second HSV cDNA encoding an HSV protein selected from the group consisting of: gC, gD, gB, UL19, and variants thereof (second HSV cDNA), wherein the first and second HSV cDNAs do not have the same sequence;
d) an upstream additional transcriptional unit (ATU) cDNA that is 5′ of the first HSV cDNA (upstream ATU cDNA);
e) a downstream ATU cDNA that is 3′ of the second HSV cDNA (downstream ATU cDNA); and
f) a furin cleavage site cDNA and 2A peptide cDNA between the first and second HSV protein cDNAs (Fur-2A cDNA);
wherein the upstream ATU cDNA, the first and second HSV cDNAs, the Fur-2A cDNA, and the downstream ATU cDNA are operably linked; and
wherein the upstream ATU cDNA, the first and second HSV cDNAs, the Fur-2A cDNA, and the downstream ATU cDNA are between P and M genes of the MV-cDNA at ATU2 or between H and L genes of the MV-cDNA at ATU3.

17. The isolated nucleic acid molecule of claim 16, wherein the first and second HSV cDNAs each encode an HSV protein sequence independently selected from the group consisting of: SEQ ID NO: 54 (HSV-2 gC, wild-type); SEQ ID NO: 57 (HSV-2 gC F327A); SEQ ID NO: 55 (HSV-2 gB, wild-type); SEQ ID NO: 58 (HSV-2 gBmut); SEQ ID NO:

58. 1 (HSV-2 gBmutdel25); SEQ ID NO: 64 (HSV-2 gBwtdel25); SEQ ID NO: 56 (HSV-2 gD, wild-type); and SEQ ID NO: 65 (HSV-2 UL19).

18. The isolated nucleic acid of claim 16, wherein the upstream ATU cDNA, the first and second HSV cDNAs, the Fur-2A cDNA, and the downstream ATU cDNA are at ATU2 or ATU3 in the MV-cDNA.

19. (canceled)

20. The isolated nucleic acid molecule of claim 16, wherein the upstream ATU cDNA sequence is set forth in SEQ ID NO: 87 or 90.

21. The isolated nucleic acid molecule of claim 16, wherein the downstream ATU cDNA sequence is set forth in SEQ ID NO: 90.

22. The isolated nucleic acid molecule of claim 16, wherein the furin cDNA of the Fur-2A cDNA encodes a protein sequence selected from the group consisting of SEQ ID NOs: 14-53, and wherein the 2A peptide cDNA of the Fur-2A cDNA encodes a protein sequence selected from the group consisting of SEQ ID NOs: 4-11.

23-49. (canceled)

50. A vector for the rescue of a recombinant measles virus, comprising the isolated nucleic acid molecule of claim 1.

51-54. (canceled)

55. A recombinant measles virus comprising in its genome a cDNA sequence comprising the nucleic acid molecule of claim 1.

56. An immunogenic composition comprising (i) an effective amount of the recombinant measles virus of claim 55, and (ii) a pharmaceutically acceptable carrier.

57. A method for treating or preventing a herpes simplex virus (HSV) infection in a subject in need thereof, comprising administering an effective amount of the immunogenic composition according to claim 56 to the subject.

58. A method for inducing a protective immune response against herpes simplex virus (HSV) in a subject in need thereof, comprising administering an effective amount of the immunogenic composition of claim 56 to the subject.

59-65. (canceled)

66. An isolated peptide comprising the sequence set forth in SEQ ID NO: 58 (HSV-2 gBmut), SEQ ID NO: 58.1 (HSV-2 gBmutdel25), or SEQ ID NO: 64 (HSV-2 gBwtdel25),
wherein SEQ ID NOs: 58.1 and 64 do not include residues 877-901 of SEQ ID NO: 55 (HSV-2 gB wild-type), or variants thereof, and
wherein SEQ ID NOs: 58 and 58.1 comprise an alanine at position 665, an alanine at position 675, and an alanine at position 677.

67-68. (canceled)

69. An isolated nucleic acid molecule encoding the isolated peptide of claim 66.

70. An immunogenic composition comprising (i) an effective amount of the isolated peptide of claim 66, and (ii) a pharmaceutically acceptable carrier.

71. A method for treating or preventing a herpes simplex virus (HSV) infection in a subject in need thereof, comprising administering an effective amount of the immunogenic composition according to claim 70 to the subject.

72. A method for inducing a protective immune response against herpes simplex virus (HSV) in a subject in need thereof, comprising administering an effective amount of the immunogenic composition of claim 70 to the subject.

73-79. (canceled)
